(19) United States
(12) Patent Application Publication
(10) Pub. No.: US 2012/0042524 A1
(43) Pub. Date: Feb. 23, 2012

(54) CAST REMOVAL DEVICE

(76) Inventors: Nick A. Deeter, Warsaw, IN (US); Robert von Seggern, Columbia City, IN (US); David W. Daniels, Winona Lake, IN (US); Mark Fox, Leesburg, IN (US); Scott Brown, Warsaw, IN (US); Christopher Bremer, Warsaw, IN (US)

(21) Appl. No.: 13/201,090

(22) PCT Filed: Feb. 11, 2010
(86) PCT No.: PCT/US10/23866

§ 371 Date: Nov. 11, 2011

Related U.S. Application Data
(60) Provisional application No. 61/151,601, filed on Feb. 11, 2009.

(51) Int. Cl. A61F 15/02; B26D 7/06; B26D 7/14

(52) U.S. Cl. 30/278; 83/13; 83/14

(57) ABSTRACT

Apparatus and methods for cutting through a layer of material. Some embodiments pertain to the removal of an orthopedic cast in a manner that is quiet and generates little or no particulate matter. In yet other embodiments there are a pair of toothed wheels that press down and penetrate into the cast and then split the cast by moving it past a sharp edge. In some embodiments the cutting head is reversible for the cutting of material in either of two opposite directions.

### CROSS REFERENCE TO RELATED APPLICATION

This application claims the benefit of priority to U.S. Provisional Patent Application Ser. No. 61/151,601, filed Feb. 11, 2009, entitled APPARATUS AND METHOD FOR REMOVING A CAST, incorporated herein by reference.

### FIELD OF THE INVENTION

The present invention pertains to methods and apparatus for cutting through a material, and in particular to methods and apparatus for cutting through an orthopedic cast.

### BACKGROUND

Casts used to set broken bones or other injuries to limbs generally consist of a hard outer shell, a sleeve, and an internal fabric or wrapping. The outer shell is typically made of layers of fiberglass or plaster. The inner wrappings are typically made from flexible woven or non-woven materials, such as cotton, polyester or other fibers. The hard outer cast shells typically are removed by using powered oscillating saws, which can be noisy and may create substantial fine debris. In order to prevent injury to patients, oscillating saws are usually operated at high frequency and low amplitude. However, oscillating saws can still cause burns or abrasions, and in many cases cause fear in many patients, especially small children.

What is needed are improved methods and apparatus for removal of a cast. Various embodiments of the present invention provide this in novel and unobvious ways.

### SUMMARY OF THE INVENTION

Various aspects of the present invention pertain to methods and apparatus for cutting a layer of material.

One aspect of the present invention pertains to an apparatus for cutting a layer of material. Some embodiments include an electric motor and a gear train receiving the output of the motor. Still other embodiments include a first wheel including a first plurality of teeth arranged about a rotational axis, the first wheel being rotationally driven by the gear train, and a foot having a top surface and a sharp edge. Rotation of the first wheel presses the material against the top surface and move the material toward the sharp edge.

Another aspect of the present invention pertains to a method for cutting a layer of material. Some embodiments include providing a first plurality of teeth arranged about an axis and a foot having a shearing surface and a top surface. Other embodiments further include engaging the material with at least one tooth from the first plurality, rotating the first pattern about the axis, moving the engaged material by rotating toward the shearing surface and above the top surface, and cutting the material with the shearing surface by rotating.

Yet another aspect of the present invention pertains to an apparatus for cutting a layer of material. Some embodiments include a wheel including a plurality of teeth equally spaced about a rotational axis, each tooth having a concave side and a convex side, the wheel being rotationally driven. The convex side of each tooth leads the concave side of the same tooth during rotation. Still other embodiments further include an elongated foot having a top surface and a sharp edge, wherein during rotation the teeth press the material against the top surface and move the material toward the sharp edge.

Still another aspect of the present invention pertains to an apparatus for cutting a layer of material. Some embodiments include a gear train having a driving member rotating in a direction about a first axis. Still other embodiments include a cutting assembly including a first wheel and a second wheel, the first wheel and the second wheel each being adapted and configured to be rotationally driven by the driving member. Rotation of the first wheel and the second wheel moves the material over a toe and toward a sharp edge. The cutting assembly is adapted and configured to be mounted on the apparatus in either of two positions, the first position being one half revolution revolved about a second axis relative to the second position, the second axis being substantially perpendicular to the first axis, for cutting material in either of two directions.

It will be appreciated that the various apparatus and methods described in this summary section, as well as elsewhere in this application, can be expressed as a large number of different combinations and subcombinations. All such useful, novel, and inventive combinations and subcombinations are contemplated herein, it being recognized that the explicit expression of each of these combinations is excessive and unnecessary.

### DESCRIPTION OF THE DRAWINGS

FIG. 1 is a side elevational view of a cast removal system according to one embodiment of the present invention. Various aspects of the figure are semi-transparent. Other aspects of the figure include modeling lines.

FIG. 2 is a front end view of the apparatus of FIG. 1 as taken along line 2-2 of FIG. 1.

FIG. 3 is a view of the apparatus of FIG. 1 with one of the housing covers removed.

FIG. 4 is a view of the apparatus of FIG. 3 with the other housing cover removed.

FIG. 5 is a view of a portion of the apparatus of FIG. 4.

FIG. 6 is a left side, top, and semi-exploded perspective of a portion of the apparatus of FIG. 5.

FIG. 6.5 is a left hand, top perspective, exploded view of a motorized cutting assembly according to another embodiment of the present invention.

FIG. 7 is a left side and frontal perspective view of a portion of the apparatus of FIG. 6, with some components shown with modeling lines and/or semi-transparent.

FIG. 8 is a frontal and left side exploded view of the apparatus of FIG. 7.

FIG. 9A is a front planar view of a portion of the apparatus of FIG. 8.

FIG. 9B is a front planar view of a portion of the apparatus of FIG. 8.

FIG. 9C is a front planar view of a portion of the apparatus of FIG. 8.

FIG. 10A is a perspective view of portions of the apparatus of FIG. 8.

FIG. 10B is an end view of portions of the apparatus of FIG. 8.

FIG. 10C is a perspective view of portions of the apparatus of FIG. 8, on the opposite side of the respective components relative to FIG. 8.

FIG. 11A is a right side elevational view of a portion of the apparatus of FIG. 8.

FIG. 11B is a top and frontal perspective view of the apparatus of FIG. 11A.

FIG. 11C is an exploded perspective view of a cutting assembly according to another embodiment of the present invention.

FIG. 11D is a bottom view of the assembled apparatus of FIG. 11C.

FIG. 11E is an end elevational view of the apparatus of FIG. 11D.

FIG. 11F is a top plan view of a portion of the apparatus of FIG. 11G as taken along line 11F-11F of FIG. 11G.

FIG. 11G is a front planer view of a portion of the apparatus of FIG. 11C.

FIG. 11H is an orthogonal cut-away view of the apparatus of FIG. 11G taken along the center line.

FIGS. 11I, 11J, and 11K are top, end, and frontal orthogonal views, respectively, of a portion of the apparatus of FIG. 11C.

FIG. 11L is a close-up of a portion of FIG. 11K.

FIG. 11M is a front planer view of a portion of the apparatus of FIG. 11C.

FIG. 11N is a cross-sectional view of the apparatus of FIG. 11M as taken along the center line.

FIG. 11O is a close-up of a portion of the apparatus of FIG. 11M.

FIG. 12 is a left side, top, and frontal perspective of a portion of the apparatus of FIG. 6.

FIG. 13 is a left side elevational view of the apparatus of FIG. 12.

FIG. 14 is a right side elevational view of the apparatus of FIG. 12.

FIG. 15 is a bottom planar view of a portion of the apparatus of FIG. 12.

FIG. 16 is a left side and frontal perspective view of a portion of the apparatus of FIG. 4.

FIG. 17 is a left side and top perspective view of a portion of the apparatus of FIG. 4.

FIG. 18 is a right side and rear perspective view of the apparatus of FIG. 17.

FIG. 19 is a left side exploded perspective view of the apparatus of FIG. 17.

FIGS. 20A and 20B are front, left side perspective views of a cutting assembly according to another embodiment of the present invention, shown both shaded only and shaded with lines, respectively.

FIGS. 21A and 21B are right side perspective views of the cutting assembly of FIGS. 20A and 20B, shown both shaded only and shaded with lines, respectively.

FIGS. 22A and 22B are left side perspective views of the cutting assembly of FIGS. 20A and 20B, shown both shaded only and shaded with lines, respectively.

FIG. 23 is an exploded, perspective view of the apparatus of FIG. 21A.

FIG. 24 is a view of the apparatus of FIG. 20A, as taken along line 24-24, except rotated 180 degrees.

FIG. 25 is a cross-sectional view of the apparatus of FIG. 24 as taken along line 25-25.

FIG. 26 is a frontal view of a portion of the apparatus of FIG. 20A.

FIG. 27 is a cross-sectional view of the apparatus of FIG. 26 as taken along line 27-27.

FIG. 28 is an enlargement of a portion of the apparatus of FIG. 26.

FIG. 29 is a view from the rear of a portion of the apparatus of FIG. 20A.

FIG. 30 is a cross-sectional view of the apparatus of FIG. 29 as taken along line 30-30.

FIGS. 31A and 31B are front, left side perspective views of a cutting assembly according to another embodiment of the present invention, shown both shaded only and shaded with lines, respectively.

FIG. 32A is a cross sectional view of a portion of the apparatus of FIG. 31A as taken in a plane through the centerline as shown by line 32-32 of FIG. 31A (housing removed).

FIG. 32B is a cross sectional view of the apparatus of FIG. 31A, as taken along a vertical plane passing through the rotational centerline, and showing schematically a material M being advanced.

FIG. 33A is an exploded perspective view of the apparatus of FIG. 31B.

FIG. 33B is a perspective view of the support of the apparatus of FIG. 31A.

FIG. 34 is a front, left side perspective view of a cutting assembly according to another embodiment of the present invention, shown shaded with lines.

FIG. 35 is a view of the apparatus of FIG. 34 with the front half of the housing removed.

FIG. 36 is a cross sectional view of the apparatus of FIG. 34 as taken along line 36-36 of FIG. 34.

FIG. 37 is a cross sectional view of the apparatus of FIG. 34 as taken in a plane represented by line 37-37 of FIG. 34 (housing removed).

FIG. 38 is an exploded perspective view of the apparatus of FIG. 34.

FIG. 39 is a perspective view of one of the advancing wheels of FIG. 38.

FIG. 40 is a perspective view of the other advancing wheel of FIG. 38.

FIG. 41 is a perspective view of a portion of an apparatus for cutting a layer of material.

FIG. 42 is a perspective view of the apparatus of FIG. 39 with the housing, keel, and one advancing wheel removed.

FIG. 43 is a side elevational cutaway view of the apparatus of FIG. 41 as taken along a vertical plane passing through the centerline.

FIG. 44 is an exploded view of the apparatus of FIG. 41.

### DESCRIPTION OF THE PREFERRED EMBODIMENT

For the purposes of promoting an understanding of the principles of the invention, reference will now be made to the embodiments illustrated in the drawings and specific language will be used to describe the same. It will nevertheless be understood that no limitation of the scope of the invention is thereby intended, such alterations and further modifications in the illustrated device, and such further applications of the principles of the invention as illustrated therein being contemplated as would normally occur to one skilled in the art to which the invention relates. At least one embodiment of the present invention will be described and shown, and this application may show and/or describe other embodiments of the present invention. It is understood that any reference to “the invention” is a reference to an embodiment of a family of inventions, with no single embodiment including an apparatus, process, or composition that must be included in all embodiments, unless otherwise stated.

The use of an N-series prefix for an element number (NXX.XX) refers to an element that is the same as the non-prefixed element (XX.XX), except as shown and described thereafter. As an example, an element 1020.1 would be the same as element 20.1, except for those different features of element 1020.1 shown and described. Further, common elements and common features of related elements are drawn in the same manner in different figures, and/or use the same symbology in different figures. As such, it is not necessary to describe the features of 1020.1 and 20.1 that are the same, since these common features are apparent to a person of ordinary skill in the related field of technology. Although various specific quantities (spatial dimensions, temperatures, pressures, times, force, resistance, current, voltage, concentrations, wavelengths, frequencies, heat transfer coefficients, dimensionless parameters, etc.) may be stated herein, such specific quantities are presented as examples only. Further, with discussion pertaining to a specific composition of matter, that description is by example only, and does not limit the applicability of other species of that composition, nor does it limit the applicability of other compositions unrelated to the cited composition. This application incorporates by reference PCT application No. PCT/US08/83453, filed Nov. 13, 2008, titled CAST REMOVAL SYSTEM.

Various embodiments of the present invention pertain to apparatus and methods for cutting a layer of material with a quiet, clean, motorized shearing (or splitting or severing) action. The apparatus and methods described herein are applicable to cutting many different types of material, such as plaster, fiberglass, wood, sheet metal, and other preferably thin layers of material. In several embodiments, there are apparatus adapted and configured for cutting and removing a plaster, cloth, fiberglass, or polyester orthopedic cast placed around a limb of a patient, or casts fabricated from any type of material and used for any purpose.

Some embodiments include an arm that extends around one side of the splitting or shearing wheel, the end of the arm having an elongated foot that extends under the wheel. In those embodiments directed toward removal of orthopedic casts, this foot is located between the splitting wheel and the patient, such that the cast material is directed between the foot and the splitting wheel. In some embodiments, the foot is elongated in the direction of travel of the material.

In some embodiments, the foot includes a shearing surface that extends upward toward the splitting or shearing wheel, and is located such that a face of the splitting or shearing wheel is in sliding contact with the shearing surface. In such embodiments the shearing action occurs by the action of the shearing sector pressing against the material that is being supported along the top of the shearing surface. In some embodiments it is advantageous for this top edge of the shearing surface to have a squared off edge having a relatively small radius of curvature, so as to support the material to be cut as closely as possible to the face of the cutting wheel. In yet other embodiments, the leading edge of the foot has a razor-type surface to assist in advance cutting of the soft material on the inside of the cast. In yet other embodiments, the heel portion of the arm (where the foot connects to the arm) has a razor-type edge for assistance in cutting the soft material that has already been split.

Yet other embodiments of the present invention pertain to a hand-held, battery operated cutting device that shears a material with a high torque, low speed shearing action. In one embodiment, the material is automatically advanced through a scissors-type shearing action at about 0.8 inches per second, although other embodiments of the present invention contemplate material flow speeds of as high as about 3 inches per second. In some embodiments the torque applied to the shearing wheel (which produces the scissors-type action) is about fifty to one hundred and fifty foot-pounds (force). It has been found that a quiet, low dust-generating shearing action within these ranges provides acceptable performance in shearing an orthopedic cast. However, other embodiments of the invention are not so constrained, and as an example, in those applications where sheet metal is sheared, the shearing speed ranges as low as about two-tenths of an inch per second.

In some embodiments, the means for automatically advancing the material is accomplished at a substantially constant velocity. Velocity is generally maintained by an electronic controller (preferably operating a software algorithm) that automatically adjusts the power provided by the motor as the toughness of the material being cut varies (such as for an orthopedic cast of varying thickness, or a layer of wood of varying thickness).

In another embodiment of the present invention, there is a hand-held, motorized shearing assembly that operates at one of a plurality of predetermined material flow velocities. In one embodiment, there is a trigger switch preferably operated by a finger of the operator. Over a first range of switch movement, the linear velocity of the shearing wheel (and in some embodiments, further of the advancing wheel) is held substantially constant at a first linear velocity. Further movement of the switch into a second, predetermined range of movement operates the shearing wheel (and possibly the advancing wheel) at a second, higher, “boost” speed. This latter, second, boosted speed can be useful in the shearing of orthopedic casts, especially when the path of the shearing wheel is relatively straight along the cast, with the slower speed being helpful when the cutting device must follow a curved path (such as for a cast that holds an arm of a patient bent at the elbow).

In another embodiment of the present invention, there is an advancing wheel having a plurality of teeth that are adapted and configured for pressing contact, and in some embodiments, penetration into the outer surface of an orthopedic cast having an external woven material. As one example, some orthopedic cast have an exterior of a cross woven fiberglass matte, with a standard spacing between adjacent threads, which thus establishes a “hole pattern” or “line pattern” in the woven material. In some embodiments, the linear distant between adjacent teeth of the advancing wheel are adapted and configured to be even multiples of this hole pattern. Such spacing increases the likelihood that as a tooth penetrates into the outer surface of the cast, and then moves the cast, that the next tooth will not necessarily fray the woven material which would be the case if the tooth pattern were not a multiple of the weave pattern. However, the present invention is not so constrained and further, various embodiments of the present invention are adapted and configured for shearing any variety of orthopedic cast material, including Goretex®.

In yet other embodiments, the apparatus includes a support member that places a downwardly extending arm with a forward extending foot around the back (aft) periphery of the shearing wheel. After the material is split by the means for shearing the material (which can be any of the shearing or splitting devices shown herein), the split material progresses aftward and goes past on either side of the arm. The forward-projecting foot reaches under the shearing wheel, such that the material flowpath (and the locus of the shearing operation occurs between the edge of the shearing wheel and the top of the foot). The bottom of the foot thus protects the patient.

In some embodiments, the foot includes a shearing surface that projects upward and is generally parallel to a face of the shearing wheel. This upwardly projecting surface has the appearing of a “shark fin” or “camel back.” Preferably, the top surface of the shark fin has a sharp, right-angle edge, so as to provide good shearing action relative to the cutting sectors. In yet other embodiments, the forward edge of the shark fin has a sharp surface, and in some embodiments a razor-type surface, for partial, advance cutting of the underside of the layer of material. In yet other embodiments, the aft portion of the foot (the “heel” or where the foot connects to the arm) is further adapted and configured to have a sharp edge, and in some embodiments, a razor-type edge, to complete, if necessary, the cutting of any soft material that was not sheared apart by the coaction of the shearing sectors against the shearing surface. In yet other embodiments, these razor-type edges are replaceable, and are held in by means such as one or more set screws.

In some embodiments, the fin extends upwardly from the top surface of the foot. As the advancing wheel moves the material over the fin and toward the shearing edge, the fin places the material in bending prior to being sheared. This bending creates a state of stress in the material that facilitates the shearing process. In some embodiments, there are advancing wheels on opposing sides of the fin, such as the fin places the top outer surface of the material being cut in a state of tension. Therefore, when the tensioned material is sheared, less shearing force is required to sever the material because of the pre-shear tensile state.

Broadly, described here are material severing devices and specifically, devices for cast removal from humans and animals. Unless otherwise stated, the term “animal” includes human beings. One embodiment described here comprises an assembly included a housing containing an electric motor, a mechanical gearing transmission component, a cutting mechanism designed to pierce and sever the cast and advance along the cast while cutting it, and a leg and foot mechanism whereas the foot extends along the underside of the cast to prevent the cutter from making contact with the skin.

The mechanical gearing transmission component would be designed to provide a slow rotation of the cutter while providing sufficient torque so as to pierce and sever a pathway down the length of a cast. While one embodiment would utilize a gearing transmission component, any means of conveying rotary motion might be utilized such as, for example, pulleys and belts or chains and sprockets. The cutter described could also provide for the deformation of the cast at the severed pathway allowing for a clear pathway, and providing easy separation of the cast upon completion of the cutting. In one variation the cutter could also shear the soft underlying wrapping through the use of increased torque combined with insertion of the cutter blade or serrations into an aperture within the foot that is traversing beneath the hard cast cover and underlying soft wrapping on a parallel path to the cutter mechanism.

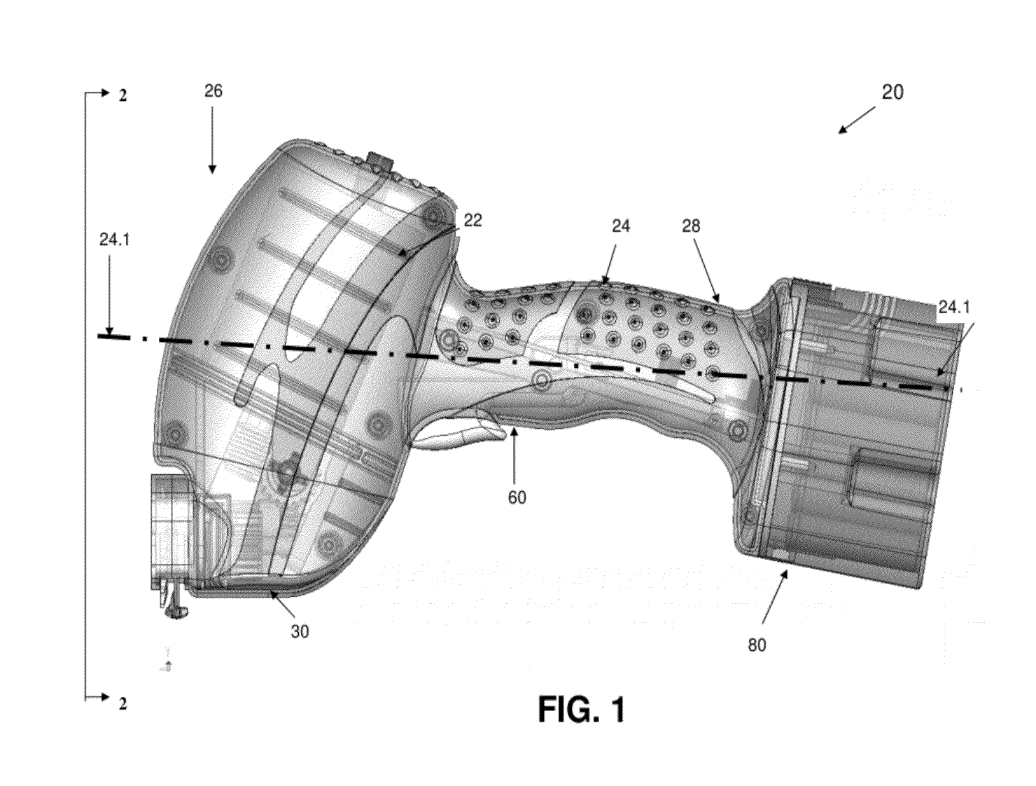

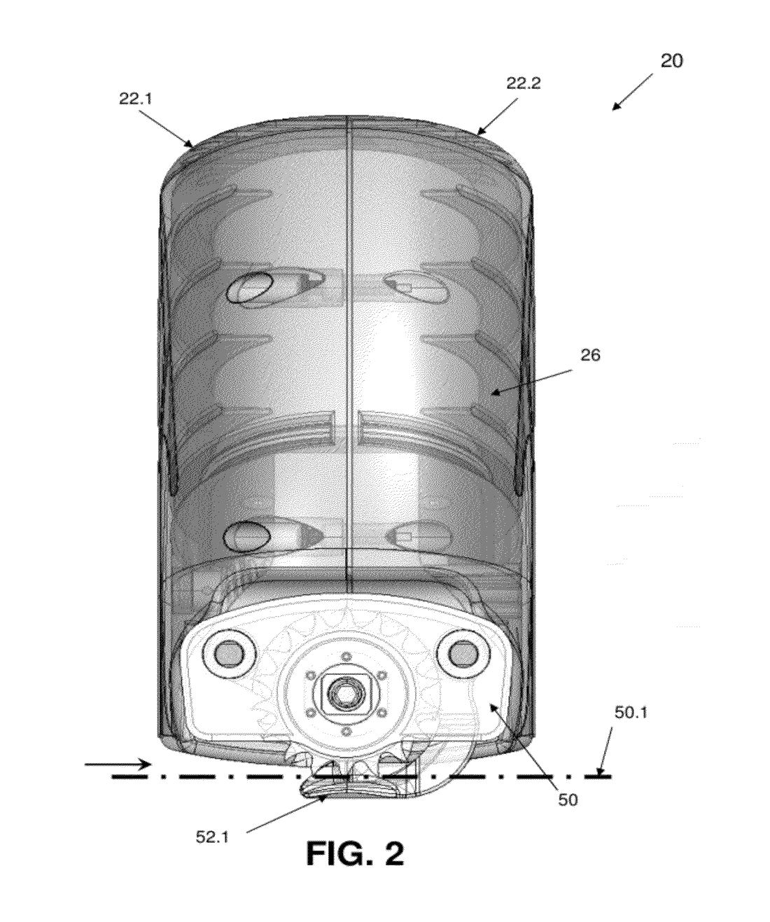

FIGS. 1 and 2 show external views of an apparatus 20 according to one embodiment of the present invention. Apparatus 20 includes a mid-positioned handle 24 adapted and configured to be grasped by the hand of a human operator. One end of handle 24 includes a battery adapter 28 that couples to a battery assembly 80. The other end of handle 24 is attached to an enclosed motorized cutting assembly 30 housed within a motor enclosure 26. As seen best in FIG. 1, handle 24 preferably includes a plurality of rounded projections for improved gripping by the palm of the operator's hand. Further, handle 24 includes various curved surfaces that are adapted and configured for improved gripping by the fingers of the operator.

As best seen in FIG. 2, a cutting assembly 50 is mounted on the front end of apparatus 20. Cutting assembly 50 preferably splits, shears, and advances a layer of material along a path 50.1. Referring to FIG. 1, handle 24 preferably establishes a support axis 24.1 that is generally orthogonal to material path 50.1. The arrow along 50.1 indicates a cutting assembly 50 oriented for movement of material in the direction of the arrow. An operator holding apparatus 20 by handle 24 is ergonomically encouraged to move apparatus 20 from side to side (from the operator's right to left or left to right). In those embodiments relating to removal of a cast from a limb of a patient, the material path 50.1 is generally parallel to the length of the limb. The orientation of axis 24.1 and 50.1 therefore permits an operator such as a surgeon, paramedic, or other health care professional to comfortably stand alongside the cast of the patient. Further, cutting assembly 50 is preferably symmetrically coupled to apparatus 20, such that the direction along path 50.1 (from right to left or from left to right) can be changed by removing cutting assembly 50, turning it around, and reattaching it to the front end of apparatus 20.

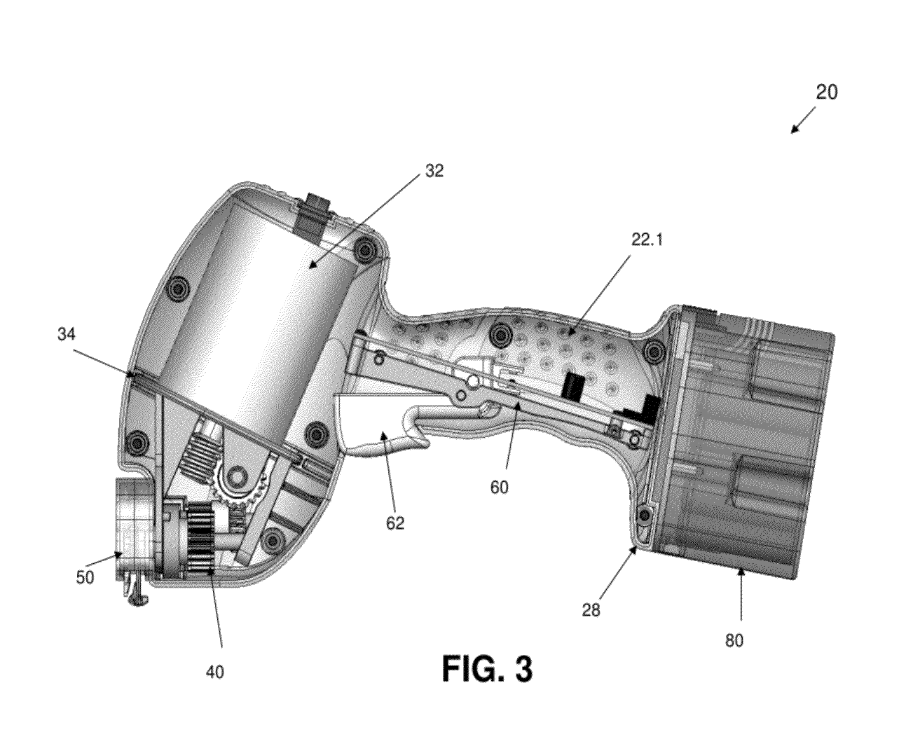

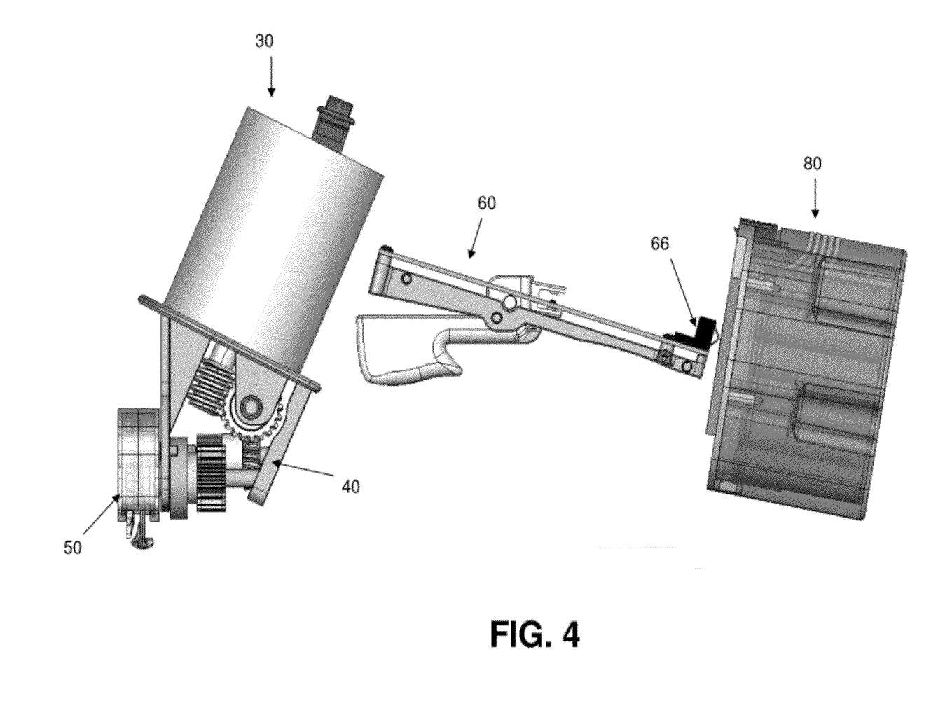

FIGS. 3 and 4 show apparatus 20 with the right and left housing covers 22.1 and 22.2, respectively, removed. Cover 22.2 is removed in FIG. 3. Covers 22.1 and 22.3 are removed in FIG. 4. Apparatus 20 includes a motorized cutting assembly 30 comprising a motor 32, gear reduction 40, and cutting assembly 50 supported by handle 24 and located in or on enclosure 26. A handle assembly 60 is located within handle 24. A battery assembly 80 is supported on one end of handle 24. Handle assembly 60 and battery 80 are in electrical communication, and power from battery assembly 80 is provided through one or more electrical contacts 66. Additionally, handle assembly 66 is in electrical communication with motorized cutting assembly 30, and provides conditioned electrical power to motor 32.

It is understood that apparatus 20 is not constrained to the placement of components shown in FIGS. 3 and 4, nor is it constrained to the type of components shown in FIGS. 3 and 4. As one example, apparatus 20 does not require a battery assembly and other embodiments of the present invention contemplate the use of electrical power from a cord that plugs into a wall socket. Additionally, the splitting and shearing apparatus and methods described herein can be powered by means other than electric motor including operation based on hydraulic power or pneumatic power, as examples.

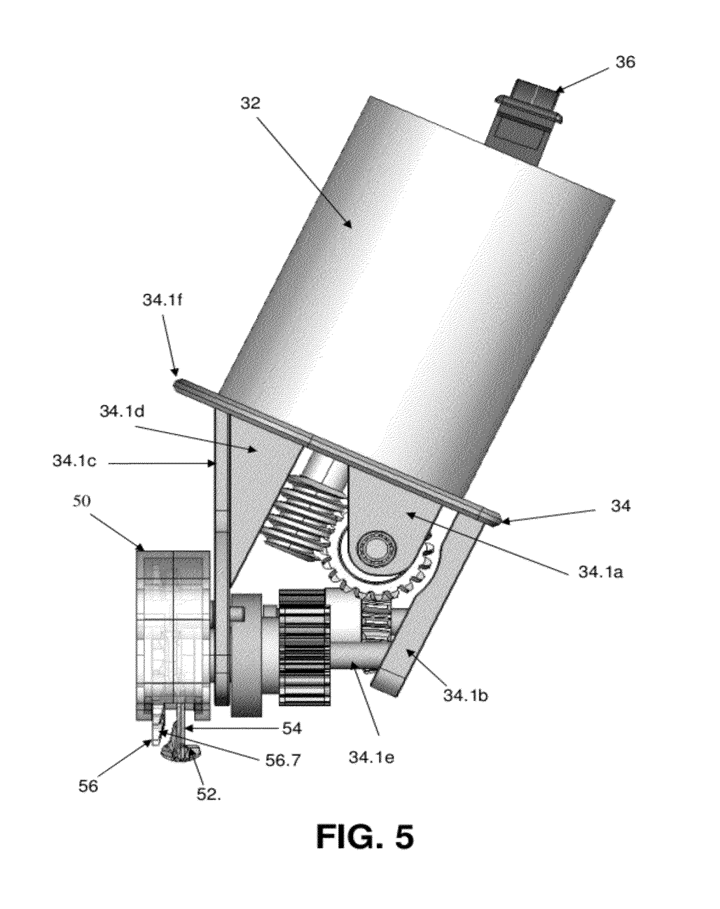

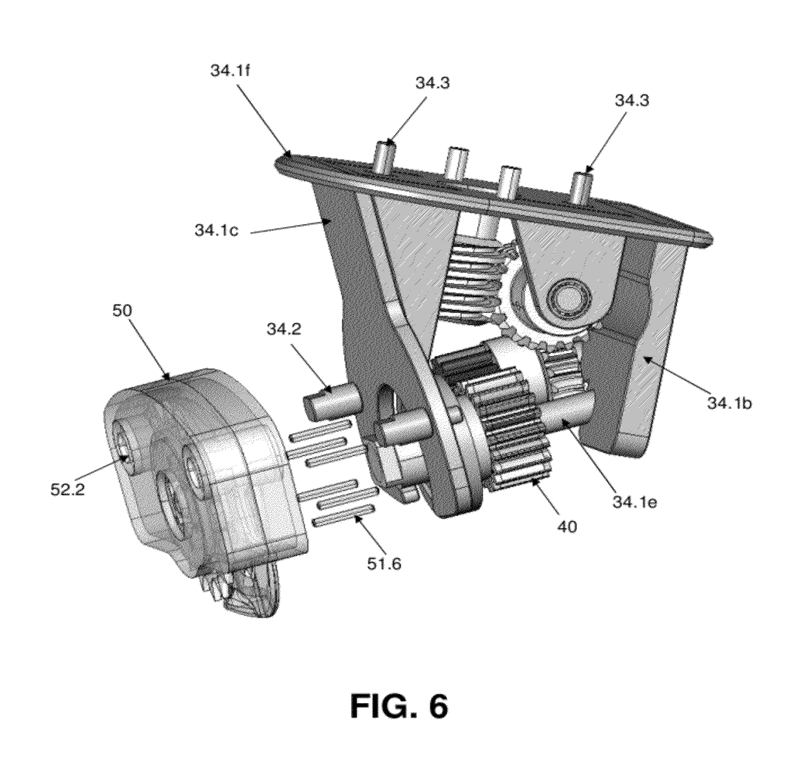

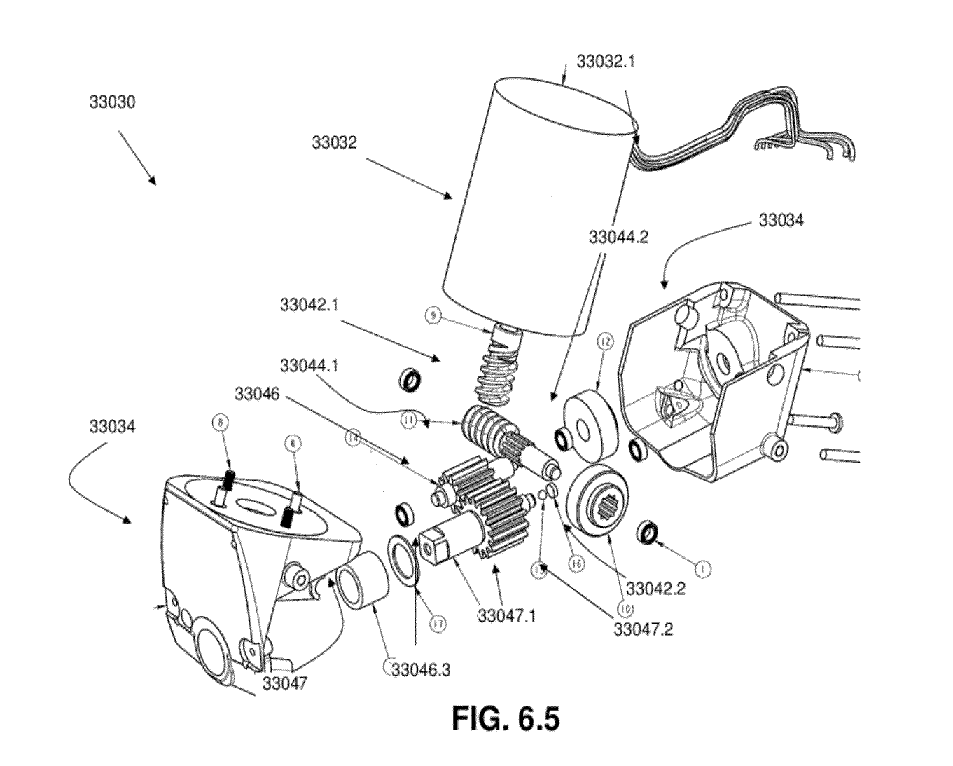

FIGS. 5 and 6 show portions of motorized cutting assembly 30. Referring to FIG. 5, motorized cutting assembly 30 includes a motor 32, gear train assembly 40 and cutting assembly 50, all supported relative to each other by a support assembly 34. Briefly referring to FIG. 3, it can be seen that support assembly 34 is located within housing halves 22.1 and 22.2 by one or more channels, or in any other manner. In one embodiment, motor 32 is a brushless DC motor, such as a model DIH 23-30-013Z as fabricated by BEI Kimco Magnetics.

Referring again to FIGS. 5 and 6, support assembly 34 includes front and rear support members 34.1c and 34.1b. A pair of triangularly shaped webs 34.1d provide bracing for front plate 34.1c relative to central platform 34.1f. A pair of projecting ears 34.1a bearingly support a portion of gear reduction assembly 40. One or more stationary axles 34.1e are coupled to either front plate 34.1c or rear plate 34.1b to bearingly support portions of gear train 40. In some embodiments, the various components of support assembly 34 are fabricated from aluminum or steel, and welded together. In yet other embodiments, support assembly 34 is fabricated from a plastic material, and the individual components are welded together ultrasonically, or adhered together, as examples. In yet other embodiments, support 34 is a one piece or multi piece molding. The aforementioned methods of fabricating support 34 are provided as examples only.

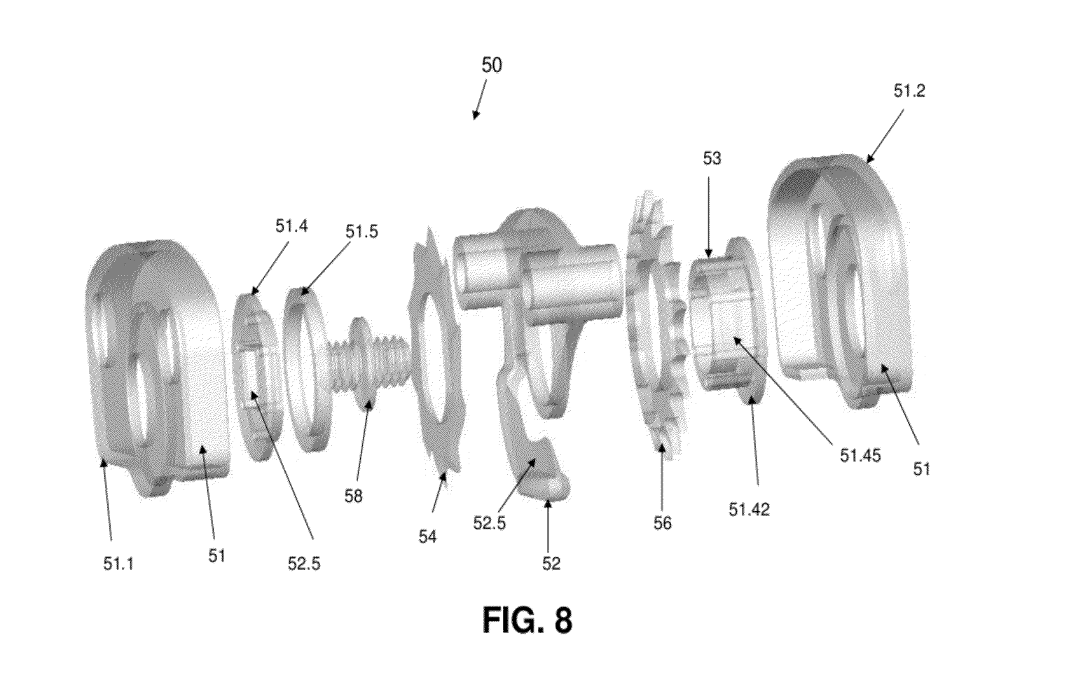

Referring to FIG. 6, a plurality of dowel pins 34.3 extend from a face of central support member 34.1f, and align the driving axis of motor 32 with the input worm gear of gear train 40. The front face 34.1c of support 34 include a pair of locating dowels 34.2 that are received within corresponding dowel holes 52.2 to align cutting assembly 50 relative to gear train 40. Also shown in FIG. 6 are a plurality of alignment pins 51.6 that align together front adapter 51.41 and rear adapter 51.42, which are shown in FIG. 8, and which will be discussed later.

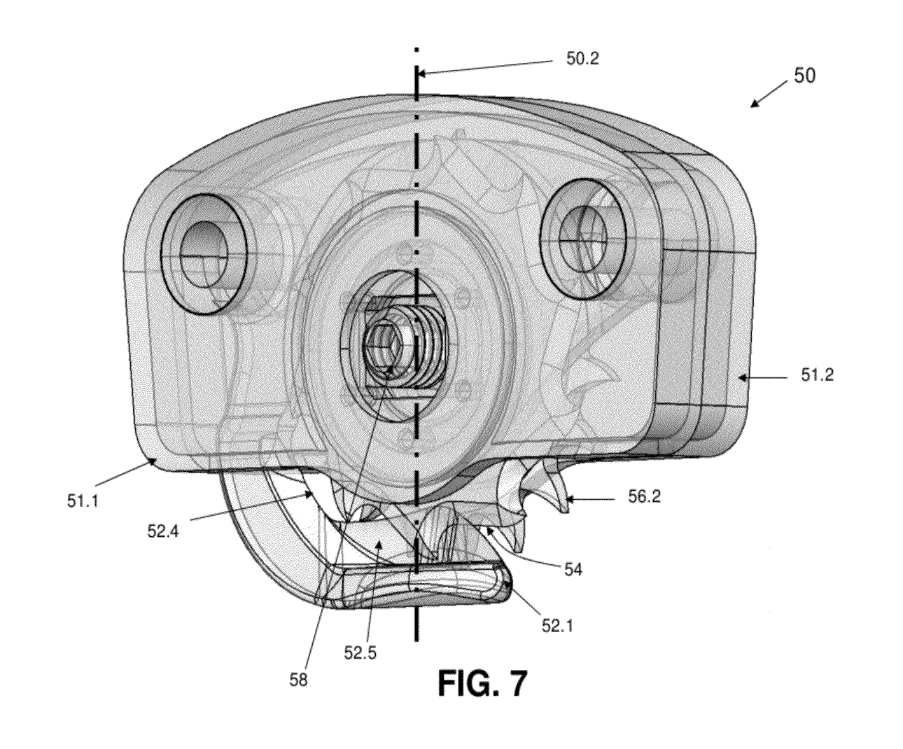

Preferably, apparatus 20 includes a switch 36 for changing the polarity of electrical power provided to motor 32. This change in polarity also changes the direction of rotation of motor 32, gear train 40, and cutting welds 56 and 54. This feature is useful in conjunction with the removal, swapping from end to end (such as about the vertical axis shown on FIG. 7), and reattachment of cutting assembly 50 so as to affect a change in the direction of material path (as previously referenced relative to FIG. 2).

FIG. 6.5 shows an exploded perspective view of a motorized cutting assembly 33030 according to another embodiment of the present invention. A brushless, DC motor 33032, having a wireloom 33032.1 providing output signals for motor sensors and further providing input power to power the motor, is coupled to a worm 33042.1 of first worm pair 33042. This first worm pair is coupled to a second worm pair 33044, which further drives a pinion set 33046. The output torque of gear train 33040 is provided at an output drive 33046.3 that is supported by a bearing 33047, which in one embodiment is a roller bearing. The output drive axis further includes a thrust ball 33047.1 and a thrust disc 33047.2 to provide an axial load on the output shaft. A pair of molded housings 33034, front and rear, provide support and enclosure for gear train 33040, as well a mounting surface for alignment and coupling of motor 33032.

FIGS. 7 and 8 show assembled and exploded views, respectively, of cutting assembly 50. A housing 51 comprising front and rear halves 51.1 and 51.2, respectively, statically retain between them a keel 52. Housing 51 and keel 52 both include alignment holes to accept dowels 34.2.

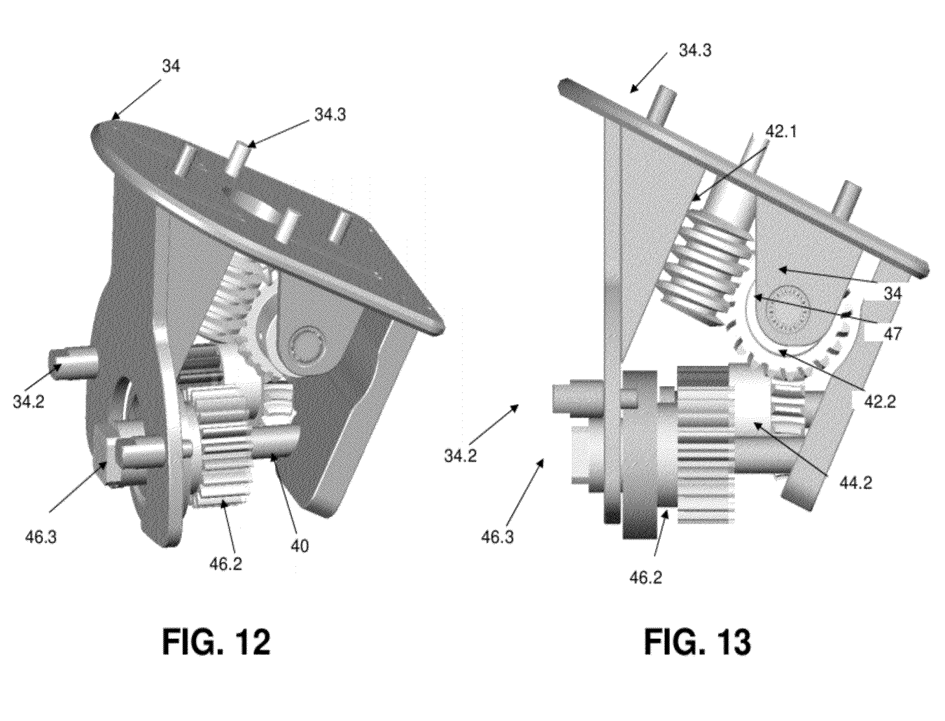

Further included within housing assembly 51 are front and rear driving adapters 51.4 aligned relative to each other by pins 51.6 previously seen in FIG. 6. Front adapter 51.41 and rear adapter 51.42 further include an interior driven interface 51.45 that have a shape complimentary to, and are driven by, adapter drive 46.3 of gear train 40 (which is shown in FIGS. 12 and 13).

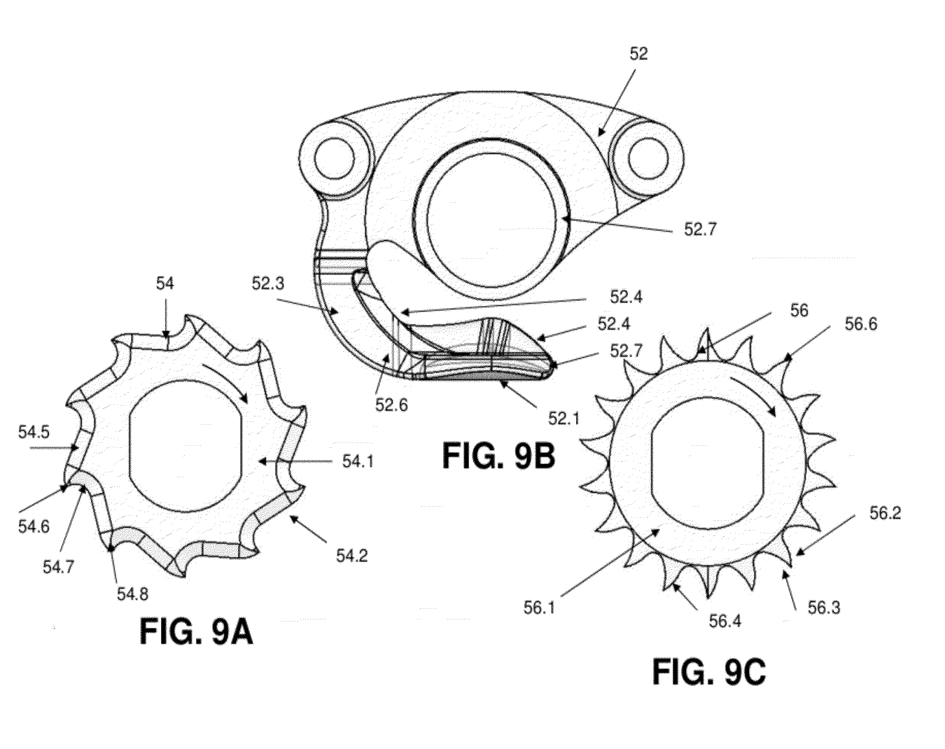

Referring again to FIGS. 7 and 8, adapters 51.4 include (either individually, or together) a cutting assembly drive surface 53 that is complimentary in shape to, and drives, driven interfaces 54.1 and 56.1 of wheels 54 and 56 (as shown in FIGS. 9a and 9c).

Cutting assembly 50 further includes a socket screw 58 comprising a threaded shaft and a centrally located cylindrically-shaped central abutment. The abutment is captured within an internal pocket formed by the coupling of front adapter 51.41 to rear adapter 51.42. The threaded portion of socket screw 58 is received within a threaded receptacle 46.4 of adapter drive 46.3 (as best seen in reference to FIG. 12, the threaded receptacle not being shown in FIG. 12). A tightening of socket screw 58 into adapter drive 46.3 results in compression of the abutment feature of socket screw 58 against an inner wall of rear adapter 51.42. If the operator desires to change the direction of material flow as indicated along path 50.1 of FIG. 2, then socket screw 58 is loosened, and adapter drive 46.3 can be removed from driven interface 51.45. Cutting assembly 50 can then be rotated 180 degrees about the vertical axis shown in FIG. 7, and realigned with dowel holes 52.2. Socket screw 58, still captured but loose within the coupled adapters 51.41 and 51.42, is then tightened such that the central abutment feature now holds adapter 51.41 in compression against adapter drive 46.3.

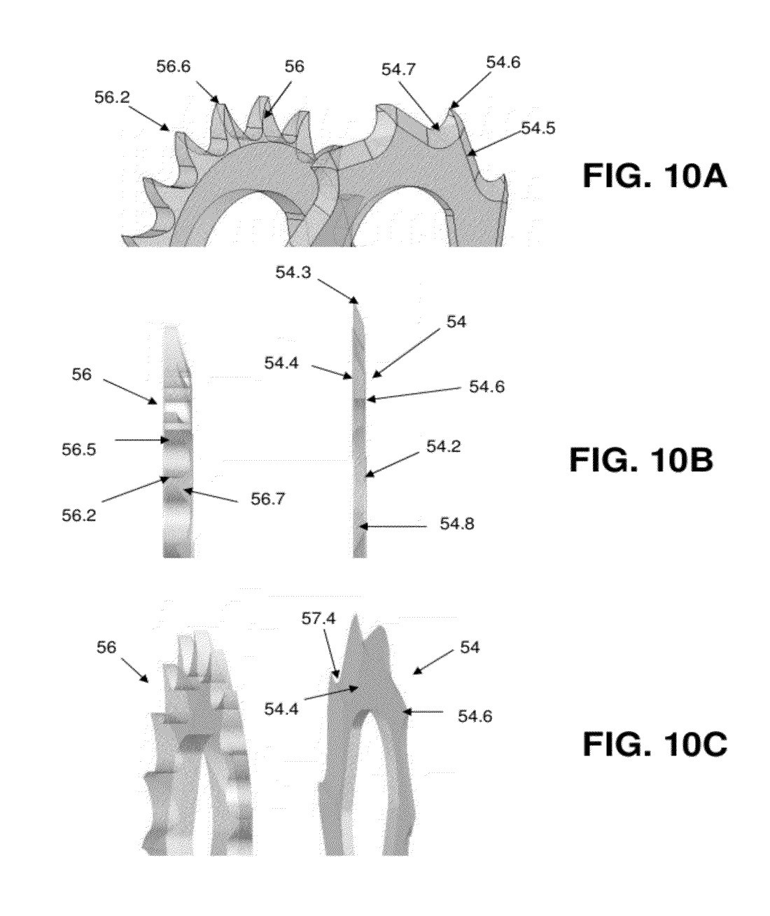

Cutting assembly 50 further includes a means for biasing shearing wheel 54 toward contact with flat surface 52.5 of keel 52. In one embodiment, and as shown in FIG. 8, a wavy spring 51.5 is placed between a ledge of front adapter 51.41, and biases a part adapter drive 51.41 and surface 54.8 of splitting wheel 54 (referring to FIG. 10b). This biasing action places a load along the rotational axis of wheel 54 so that flat surface 54.4 of wheel 54 is in sliding contact with flat surface 52.5 of keel 52. As best seen in FIGS. 10A and 10B, wheel 54 has a sharp-edged perimeter 54.3 that extends around flat surface 54.4.

FIGS. 9, 10, and 11 show various views of the splitting or shearing wheel 54, the advancing wheel 56, and keel 52. The splitting wheel 54 is shown in FIGS. 9A, 10A, 10B, and 10C. Wheel 54 includes a plurality of shearing or splitting sectors 54.2 arranged in a generally cylindrical pattern. Preferably, there are anywhere from about 4 to about 14 sectors equally spaced around the periphery of the wheel. Each sector preferably includes a splitting edge 54.5 which, for the embodiment shown in FIG. 9A, is preferably linear. The beginning of the shearing sector includes a concave transitional section 54.7 that begins (taking into account the direction of rotation shown on FIG. 9A) with a lead in portion that extends radially inward toward the center of the wheel 54. Transitional section 54.7 then curves from the radially inward direction to alignment with splitting edge 54.5. Edge 54.5 is canted aft an outward angle relative to its tangency with the end of transitional section 54.7. Splitting edge 54.5 thereby has a component of motion (when wheel 54 is rotating) that is generally radially outward from the center of wheel 54. Each splitting sector 54.2 is closed out with a section 54.8 that transitions from a point of tangency with the end of splitting sector 54.5 toward a tip or apex 54.6. From apex 54.6, the next splitting sector 54.2 begins with the concave transitional portion 54.7. Wheel 54 further includes a driven interface 54.1 for rotating wheel 54 and transmitting the torque required to shear the layer of material. As best seen in FIGS. 10B and 10C, wheel 54 includes a substantially flat surface 54.4 that, during operation, slides against flat surface 52.5 of keel 52. The side of wheel 54 opposite of flat surface 54.4 has a surface adapted and configured to receive a biasing force from spring 51.5.

Advancing wheel 56 can be seen in FIGS. 9C, 10A, 10B, and 10C. Wheel 56 has a driven interface 56.1 adapted and configured to receive a rotational input and a torque input from an adapter 51.4. Wheel 56 includes a pattern of teeth 56.2 that are arranged in a generally cylindrical pattern about the center line of wheel 56. In one embodiments, each tooth includes a generally convex-shaped side 56.4 that meets a concave-shaped side at an apex or tip 56.6. As best seen in FIGS. 10A and 10B, each tooth in pattern 56.2 is asymmetrically shaped, as best seen in edge-on-view FIG. 10B. Each tooth 56.2 has a surface 56.7 that is spaced away from the keel (as best seen in FIGS. 1 and 5). The side 56.5 opposite of the keel side is substantially flat. Referring to FIG. 5, wheel 56 has teeth 56.2 that are axially spaced apart from the portion of the material being split. This axial spacing is adapted and configured to provide that each tip 56.6 is able to come into contact with portions of the material being cut that are not at the frayed or weakened split line of the material. However, other embodiments of the present invention contemplated advancing wheels having teeth that are symmetric, or biased toward the keel. However, some of these embodiments are adapted and configured to provide spacing between the split interface of the material and the portion of the material being contacted by advancing wheel 56, so that advancing wheel 56 is able to contact a portion of the material strong enough to advance the material.

In one embodiment, the tips 56.2 have sharp edges. Further, the axis of wheel 56 is located relative to the path of material along foot 52 such that the tips 56.2 press firmly against the surface of the material. In some embodiments, teeth 56.2 make indentations on the material as it is driven. In other embodiments, teeth 56.2 penetrate the top surface of the material. Further, yet other embodiments of the present invention contemplate the use of an advancing wheel 56 that relies on friction to advance material being cut. In one such embodiment, wheel 56 includes a rubber coated periphery that comes into frictional contact with the surface of the material. In yet other embodiments, the periphery of wheel 56 has a plurality of ridges which improves the frictional contact by establishing a frictional contact patch that is a narrow contact line.

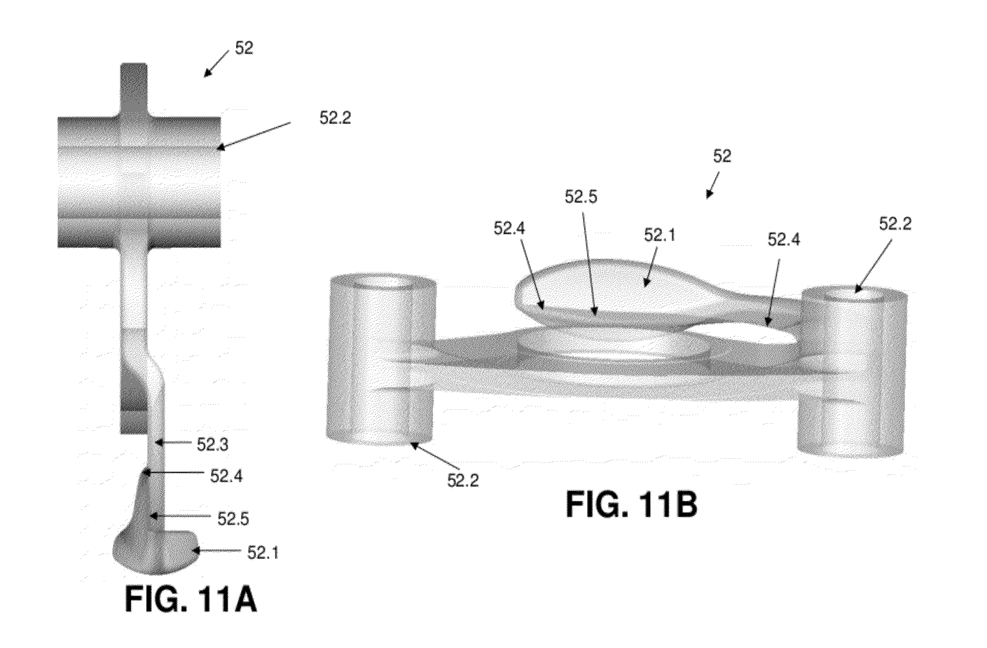

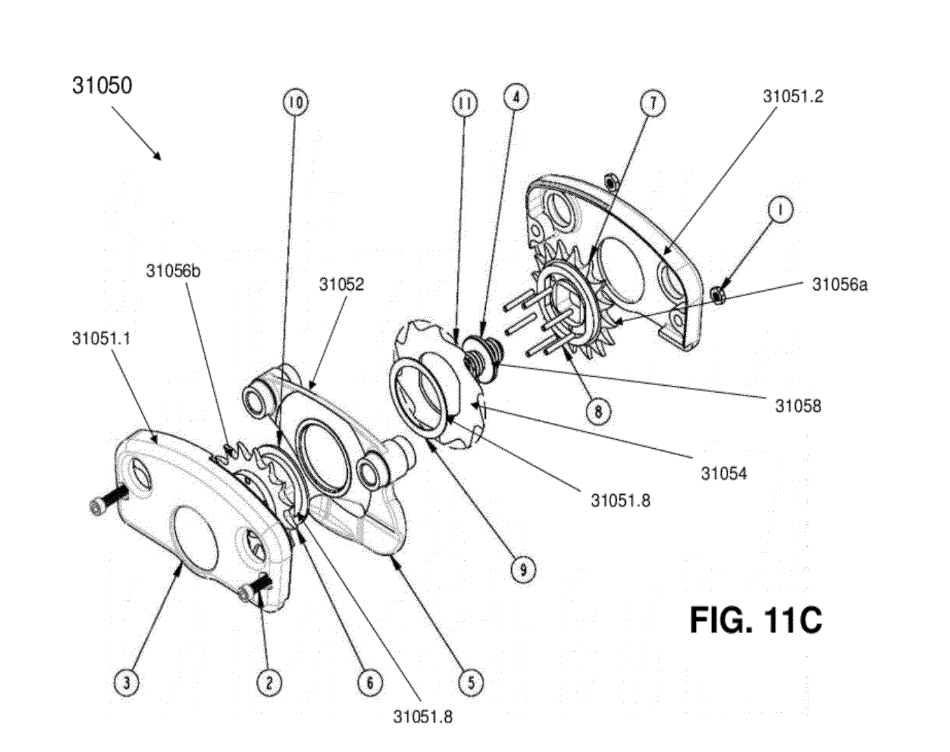

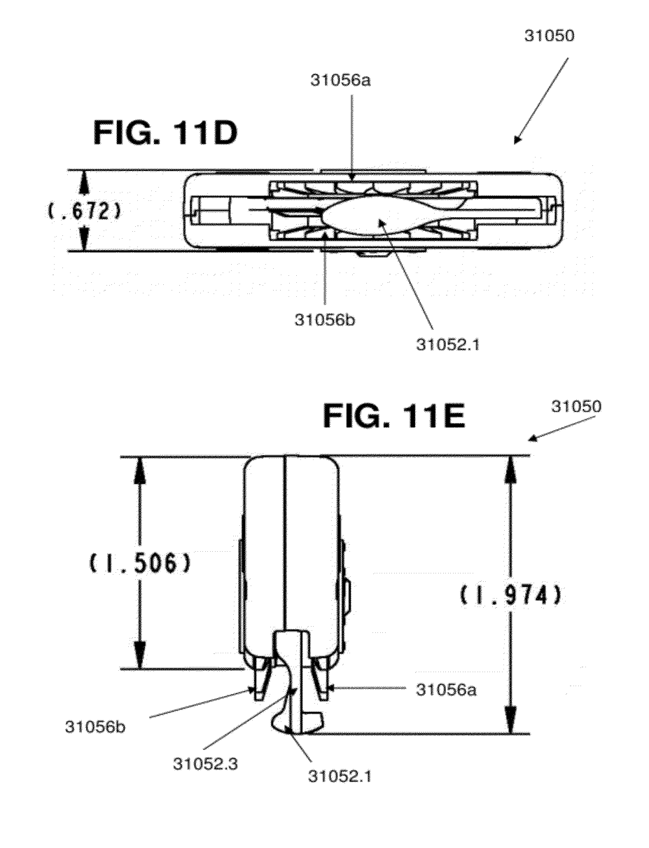

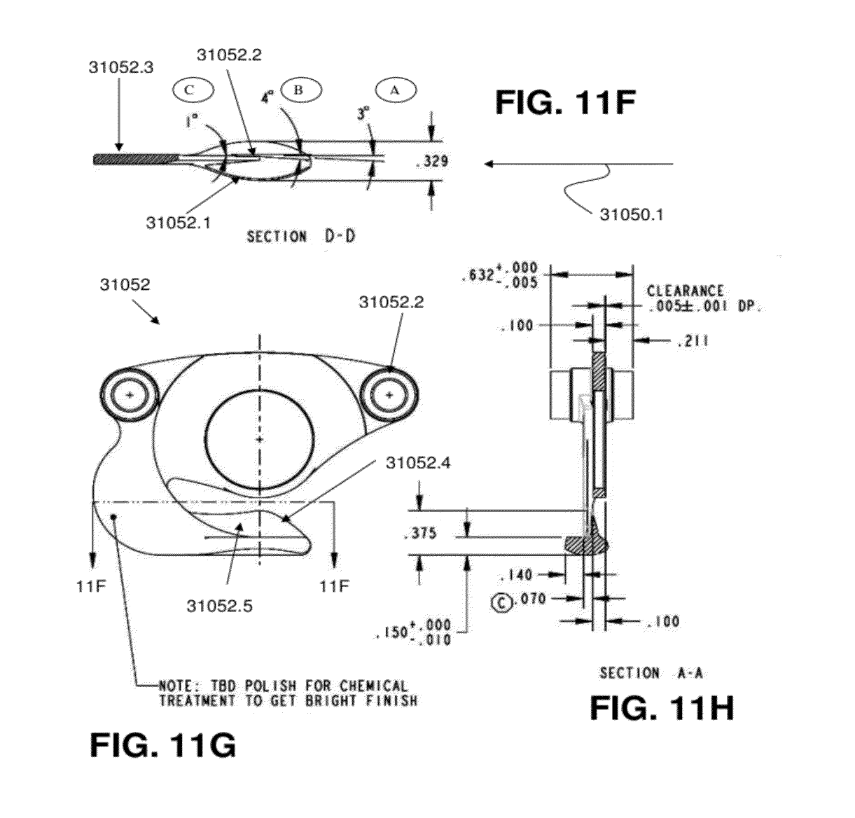

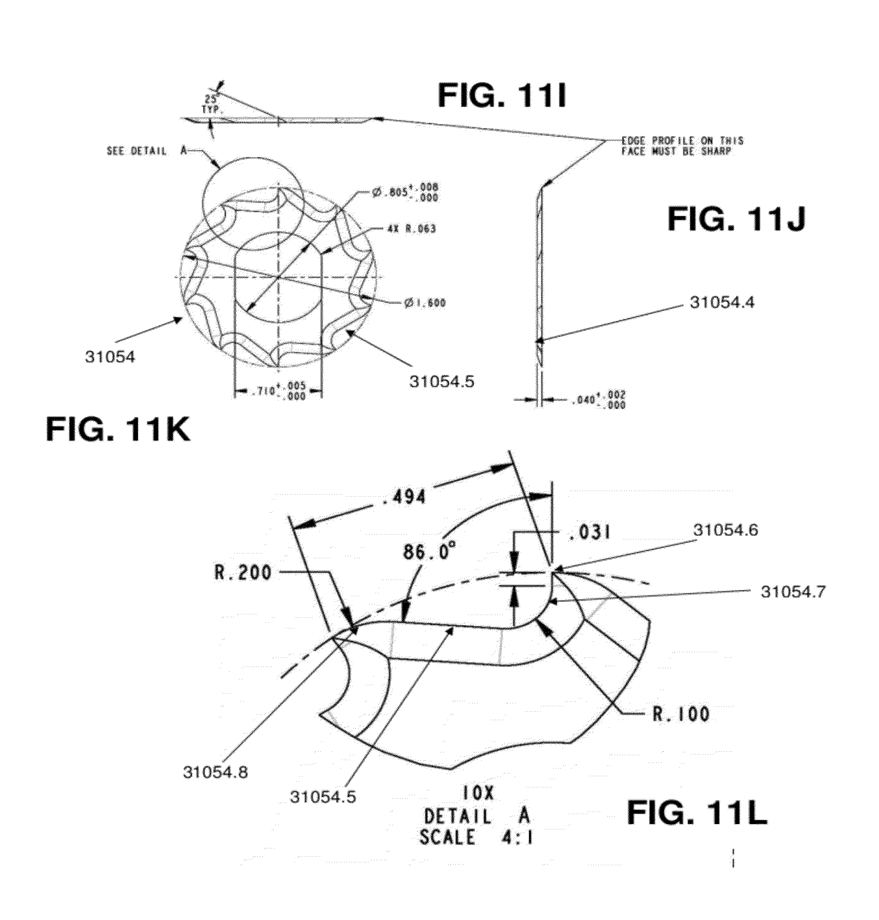

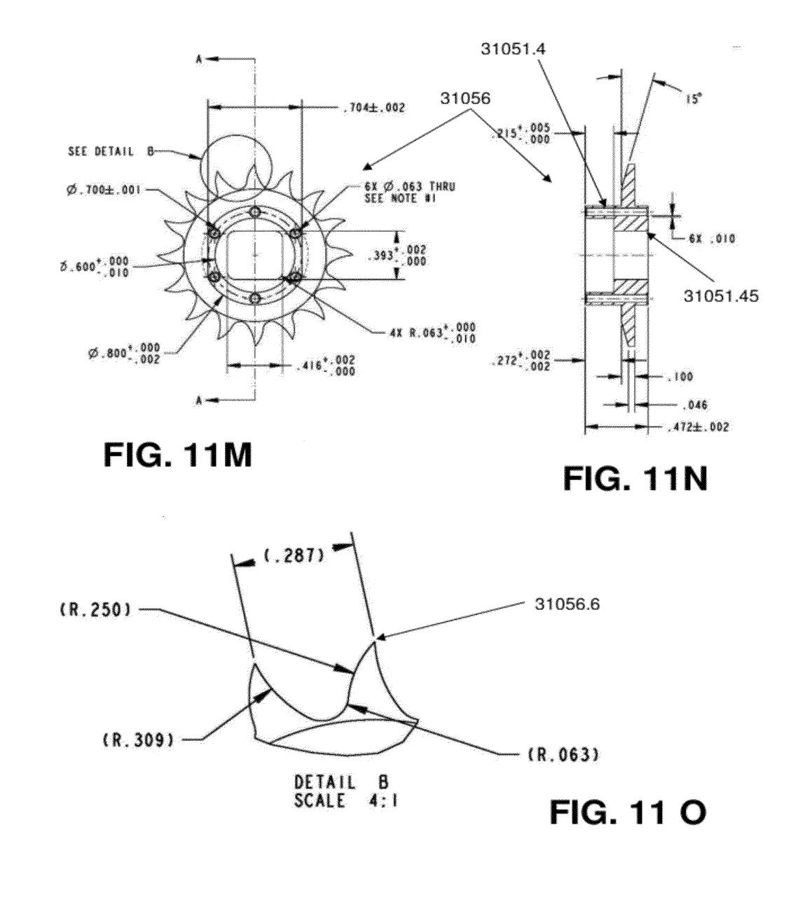

Keel 52 can be seen in FIGS. 9b, 11a and 11b. Keel 52 includes a pair of dowel holes 52.2, each on an opposite side of a central clearance hole 52.7. Adapters 51.4 extend through clearance hall 52.7 to drive wheels 54 and 56. A central structural web extends in between each dowel hole 52.2 and central passage 52.7. Also extending from this central structural web is an arm 52.3 that extends downward toward the material path, and further supports a foot 52.1 adapted and configured to be located under the material path. As best seen in FIG. 11a, arm 52.3 jogs to the right (as shown in FIG. 11a). This offsetting jog permits arm 52.3 to have on it a sharp edge 52.4 that performs any final shearing of fibers not otherwise sheared or split by wheel 54.

Foot 52.1 has a substantially rounded and smoothed underside so as to not cause abrasions when this underside passes over a patient's skin, for those embodiments in which apparatus 20 is used as a cast removal device. The present invention also contemplates those embodiments in which an inventive apparatus is used to shear through paper, wood, sheet metal, or fabric, and in this embodiment the underside of foot 52.1 does not have to be rounded or curved.

As best seen in FIGS. 11a and 11b, a “shark fin” or flat surface 52.5 extends upwardly from the topside (material side) of foot 52.1. One side of this projection has a substantially flat surface 52.5 that is adapted and configured to be in sliding contact with wheel surface 54.4, and further to co-act with wheel 54 to split or shear the layer of material. In some embodiments, sharp edge 52.4 extends from arm 52.3 forward (opposite of the direction of the material flow) and along the upper edge of flat surface 52.5. However, in yet other embodiments, the top surface of flat surface 52.5 is not a sharp edge.

Referring to FIGS. 11a and 11b, dowel holes 52.1 have a length that is adapted and configured to provide rigidity and precision in the mounting of cutting assembly 50 to dowels 34.2.

FIGS. 11C to 11O depict various views of a cutting apparatus 31050 according to another embodiment of the present invention. Cutting assembly 31050 is similar to assembly 50, but with several changes. Assembly 31050 includes first and second advancing wheels 31056a and 31056b, preferably stationed on opposite sides of splitting wheel 31054. In addition, cutting assembly 31050 does not include a wavy spring for biasing the position of the splitting wheel. Further, each advancing wheel 31056a and 31056b incorporates apparatus similar in function to driving adaptors 51.4.

As best seen in FIGS. 11D and 11E, advancing wheels 31056a and 31056b are arranged on opposite sides of splitting wheel 31054 and further on opposite sides of arm 31052.3 of keel 31052. The teeth of each advancing wheel are preferably displaced outwardly from the plane in which the material is cut, as best seen in FIG. 11E. By spacing the ends of the advancing teeth away from the cut, there is less chance of the advancing teeth pressing against, and in some embodiments penetrating, the surface of the material too close to the frayed or weakened cut (split) edges of the material. However, the present invention is not so constrained, and further contemplates those embodiments in which the advancing teeth are roughly centered about a central plane of the corresponding advancing wheel, and also those embodiments in which the teeth are splayed inward toward the plane of the cut.

FIGS. 11F, 11G, and 11H depict various views of keel 31052. As seen best in FIG. 11F, the shearing face 31052.5 of keel 31052 has a multifaceted face. A lead-in portion 31052.5A is angled such that it falls away from the flat face 31054.4, with reference to the direction 31050.1 of motion. A second facet of the flat surface of foot 31052.5 falls further away from contact with wheel 31054, in an intermediate planar faceted section 31052.5B. The distal-most portion of the shearing surface 31052.5 of foot 31052.1 is a third angled surface 31052.5C that falls away at an angle less steep than angles A or B. In one embodiment, the first faceted surface is angled three degrees falling away from the advancing wheel. The intermediate cutting facet falls away by about four degrees. The final planar facet C falls away at about one degree. In one embodiment, the angular orientation of portion 31052.5C helps create interference at the shearing interface to increase contact pressure between the face of the cutting wheel and the keel foot. The angular orientation of surface 31052.5A establishes an angle at which the cutting sectors meet the top surface of the foot, and helps to create improved shearing action relative to the soft casting materials (such as the woven materials).

FIGS. 11I, 11J, 11K, and 11L depict various views of a cutting wheel 31054 according to one embodiment of the present invention. Referring to FIG. 11L, a single cutting sector 31054.2 can be seen in detail. In one embodiment, each cutting sector begins (relative to the flow of material) from an apex 31054.6 for a short linear span of about three one-hundredths of an inch, and then blending tangentially into a transitional portion 31054.7 that is curved concave inwardly, and in some embodiments has radius of curvature of about one-tenth of an inch. This partly linear, partly curved transitional section 31054.7 tangentially blends into a substantially linear shearing section 31054.5 that, in one embodiment, is angled generally perpendicularly relative to the tip by less than about ninety degrees. The generally linear cutting section 31054.5 tangentially transitions to a curved close out section 31054.8 that is preferably curved concave outward, and ends in the tip 31054.6 of the next section.

FIGS. 11M, 11N and 11O depict various views of an advancing wheel 31056 according to another embodiment of the present invention. Wheel 31056 incorporates integrally a driving adaptor 31051.4. Wheel 31056 further incorporates a driven interface 31051.45 complementary in shape to a corresponding driving member 31046.3.

FIGS. 12, 13, 14, and 15 show various views of support assembly 34 and gear reduction assembly 40. Gear reduction assembly includes a first worm drive 42 that drives a second worm drive 44. Speed and torque from the output of second worm drive 44 is provided to a pinion pair 46, and finally to an adapter 51.4.

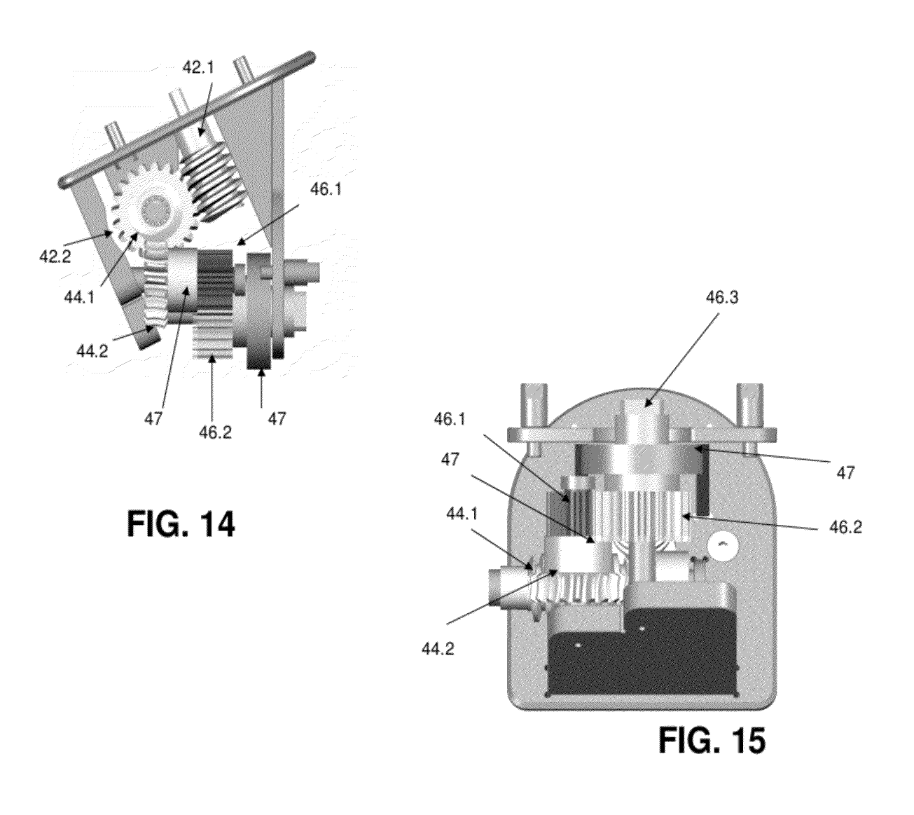

As seen in FIGS. 13 and 14, a rotational speed and torque from motor 32 is provided to a first worm gear 42.1. Worm gear 42.1 is in engagement with the corresponding worm wheel 42.2, the latter supported by support 34. In one embodiment, worm gear 42.1 is preferably fabricated from a first, harder material, and in one particular embodiment is fabricated from steel of the grade SAE 1144. In that embodiment, the worm 42.1 has an axial pitch of 0.133; has 2 threads; is a right hand helix with a lead angle of 10.9 degrees; a pitch diameter of 0.44 inches; a major diameter of 0.53; a minor diameter of 0.36 (all diameters in inches); and a pressure angle of about 20 degrees. The driven worm wheel 42.2 is preferably fabricated from a second material that is not as hard as worm 42.1, and in one embodiment worm gear 42.2 is fabricated from a plastic material such as nylon 66. In one embodiment, worm wheel 42.2 has a diametral pitch of 23.57; a helix angle of 10.8 degrees; is a right hand helix; has a pitch diameter of about 0.89 inches; a major diameter of about 0.96 inches; a minor diameter of 0.79 inches; and a pressure angle of about 20 degrees. In one embodiment, worm gear 42.2 is coupled to its shaft by sliding splines.

Referring now to FIGS. 14 and 15, located on the same shaft with worm wheel 42.2 is a second worm gear 44.1. Worm gear 44.1 engages a worm wheel 44.2. Worm wheel 42.2 is supported on a shaft along with a pinion drive gear 46.1 (with a bearing being placed in between these gears). In one embodiment, the second worm gear 44.1 is fabricated from a first, harder material, such as SAE 8620 and is case hardened in a specific embodiment, worm 44.1 has an axial pitch of about 0.16; has one thread; has a right hand helix; a helix angle of about 6.7 degrees; a pitch diameter of about 0.43; a major diameter of about 0.53; a minor diameter of 0.30; and a pressure angle of about 20 degrees. In one embodiment, the shaft that incorporates worm 44.2 includes a splined section to accept worm gear 42.2. Worm wheel 44.2 is preferably fabricated from a softer material than worm 44.1, and in one embodiment worm wheel 44.2 is fabricated from CA 673 bronze. In one specific embodiment, worm gear 44.2 has a diametral pitch of about 19.9; has 18 teeth; is a right handed helix; has a helix angle of about 6.7 degrees; a pitch diameter of about 0.91; a major diameter of about 0.98; a minor diameter of about 0.75; and a pressure angle of about 20 degrees.

Pinion drive gear 46.1 in turn drives a larger pinion driven gear 46.2, as best seen in FIGS. 12 and 15. The driven pinion member 46.2 is located on the same shaft with adapter drive 46.3, with a bearing 47 being interposed there between. In one embodiment, the overall gearing ratio from the output speed of the motor to the input speed of the motor to the output speed of drive 46.3 is about 330:1, such that gear train 40 has an output speed that is less than the input speed of motor 32, and is driven with an output torque that is greater than the input torque of motor 32.

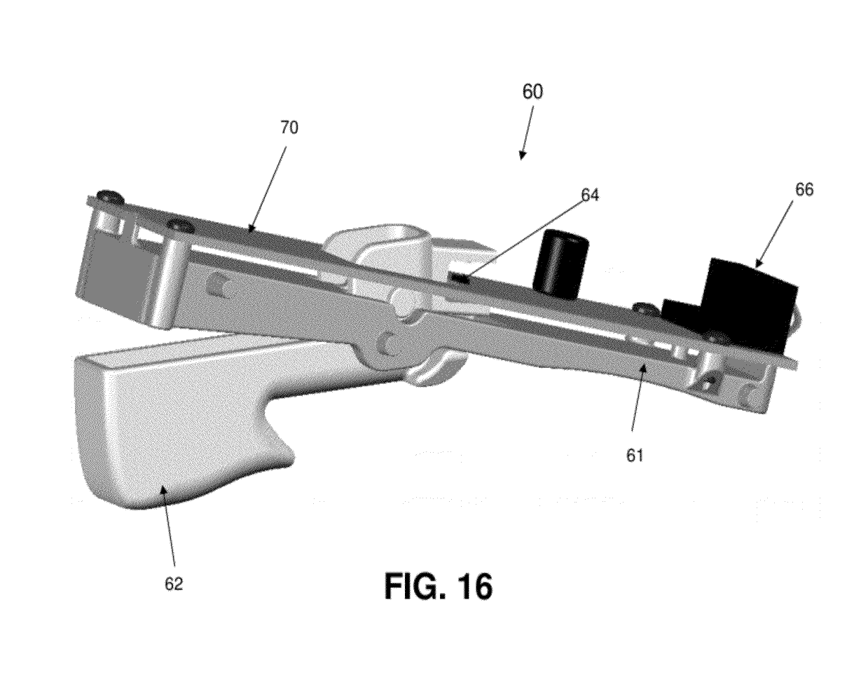

FIG. 16 is a prospective view of a handle assembly 60 according to one embodiment of the present invention. Handle 60 includes a structural chassis 61 that supports a pivotal finger switch 62. The interior end of finger switch 62 includes a pair of projections, each having on their surface a magnet. Located in between the magnets on these projections is a Hall sensor 64, located on a circuit card 70. A plurality of electrical contacts 66 located at one end of circuit card 70 provide an input for electrical power to circuit card 70.

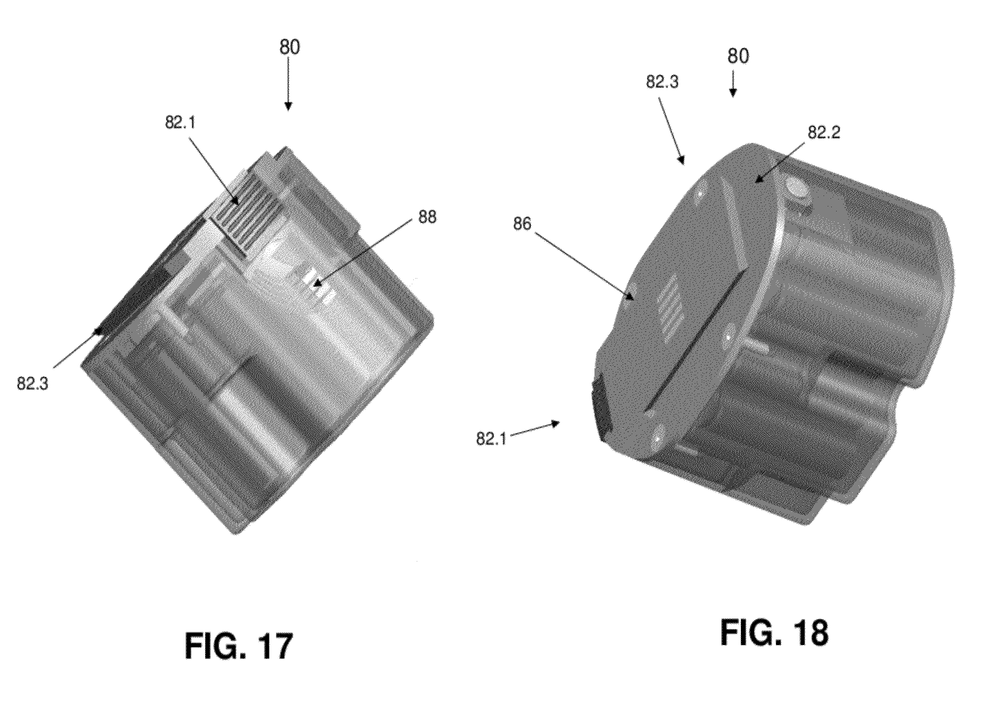

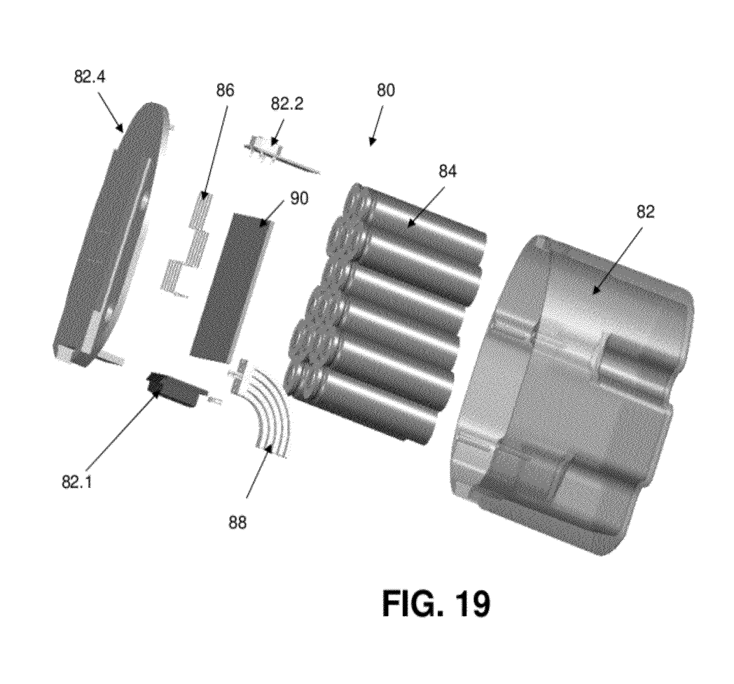

FIGS. 17, 18, and 19 are various views of a battery assembly 80 according to one embodiment of the present invention. Battery assembly 80 includes a plurality of batteries 84 located within a housing 82. In one embodiment, the batteries are of a nickel-metal hydride type. Power from the batteries is provided to a circuit card 90 that conditions the power as required. A plurality of electrical contacts 86 provide battery power to contacts 66 of handle assembly 60. An assembly of five LEDs and corresponding light pipes 88 receive a signal from circuit card 90 pertaining to the state of charge of batteries 84. Referring to FIG. 17, the LEDs 88 indicate to the operator whether or not there is sufficient charge to sever the cast of another patient.

Cover 82.4 of housing 82 includes dovetail grooves 82.3 that are grasped by complimentary-shaped grooves on the underside of battery adapter 28. Battery assembly 80 further includes a spring-loaded sliding switch 82.1 that locks battery assembly 80 to handle 24. A button 82.2 provides an actuatable switch by which the operator can request the status of batteries 84 to be displayed on LEDs 88.

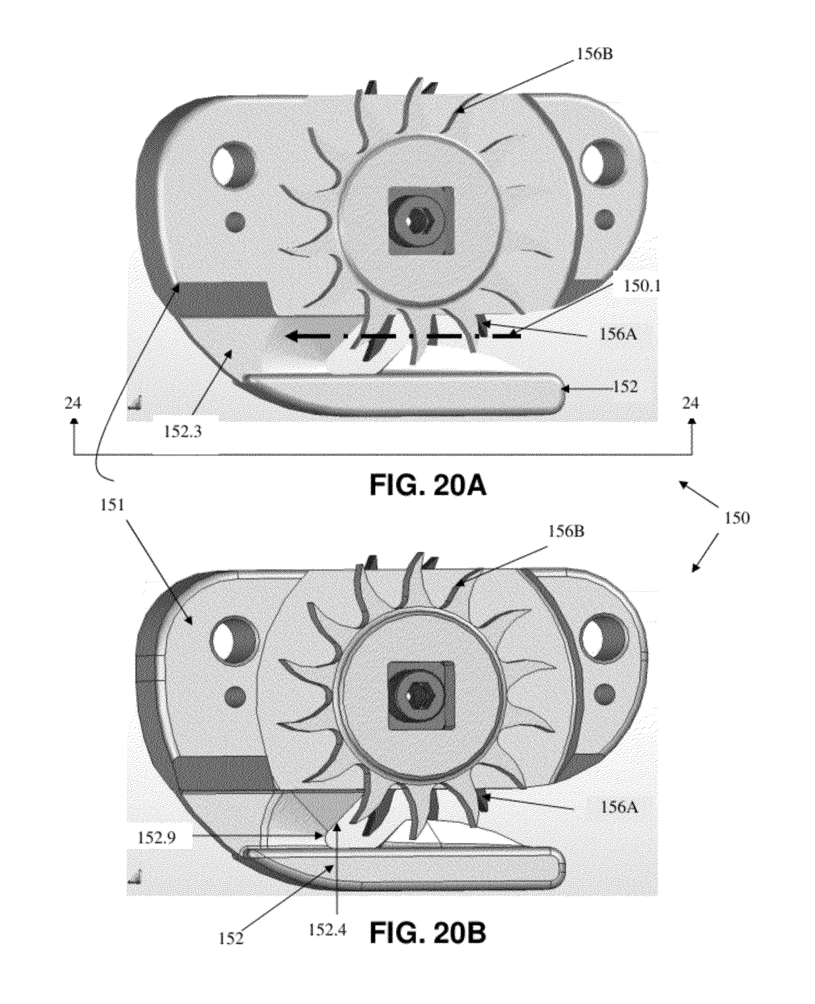

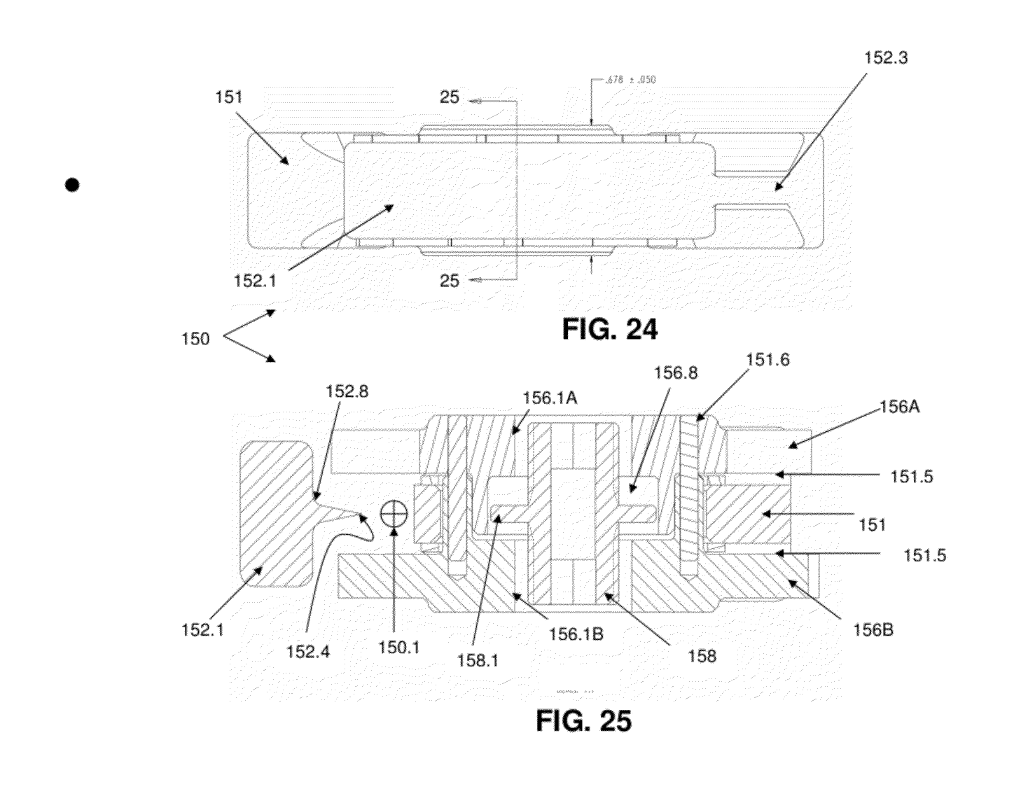

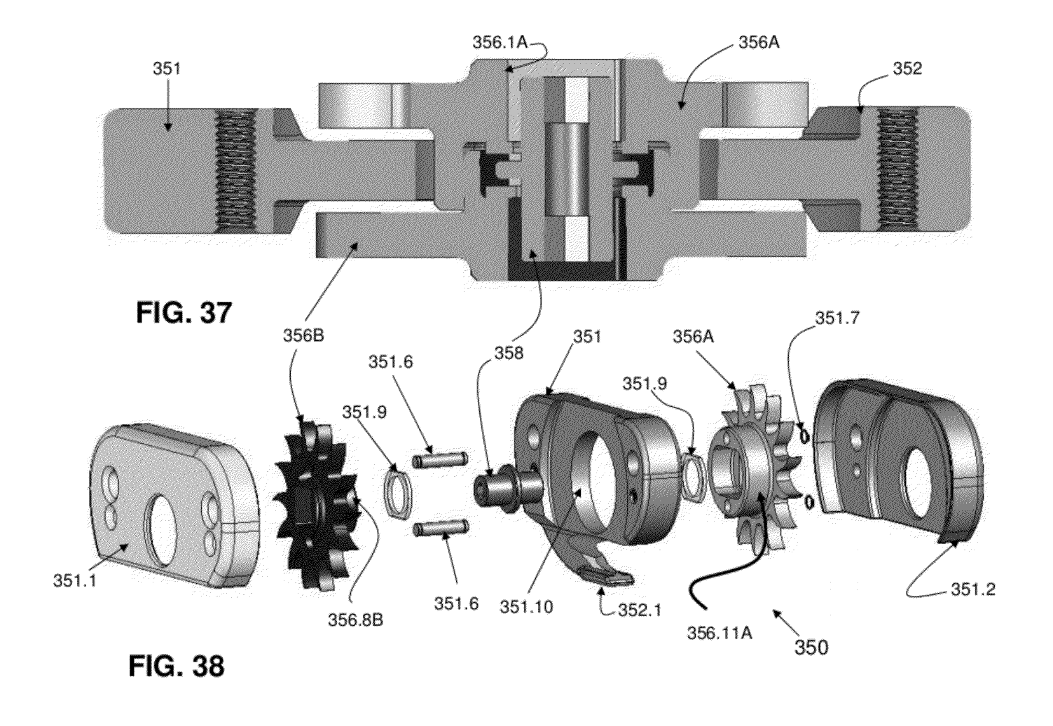

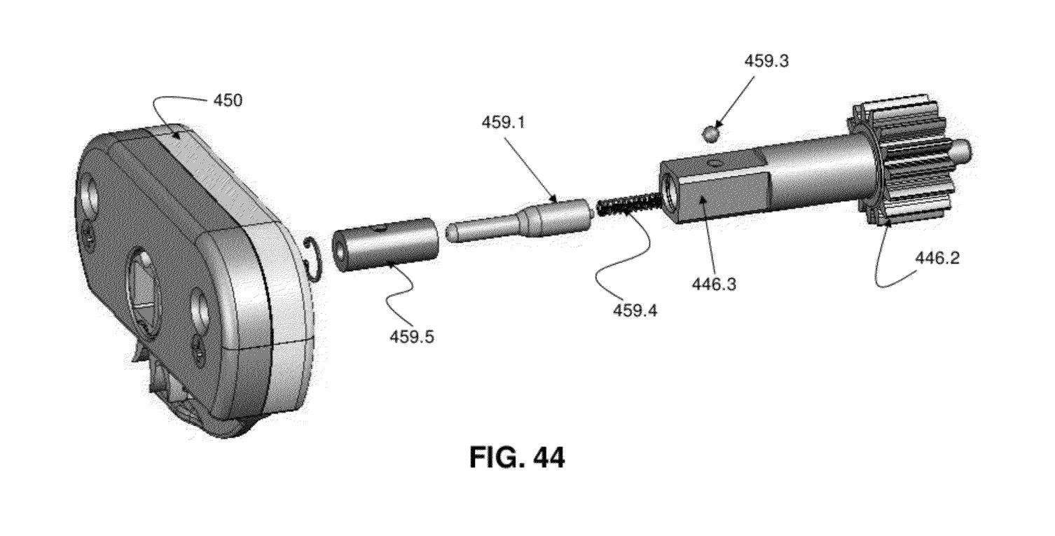

FIGS. 20-44 are scaled drawings, although one of ordinary skill in the art will appreciate that the scale can change from one figure to another. Further, it is appreciated that these drawings represent specific embodiments, and the relative dimensions of one feature to another are not to be construed as limiting.

FIGS. 20-30 depict a cutting assembly 150 according to another embodiment of the present invention. Cutting assembly 150 is the same as the cutting assemblies previously described, except for the changes shown and described hereafter. As one example, cutting assembly 150 is usable with cutting apparatus 20 previously described, and in some embodiments, cutting assembly 150 can be directly substituted for cutting assembly 50.

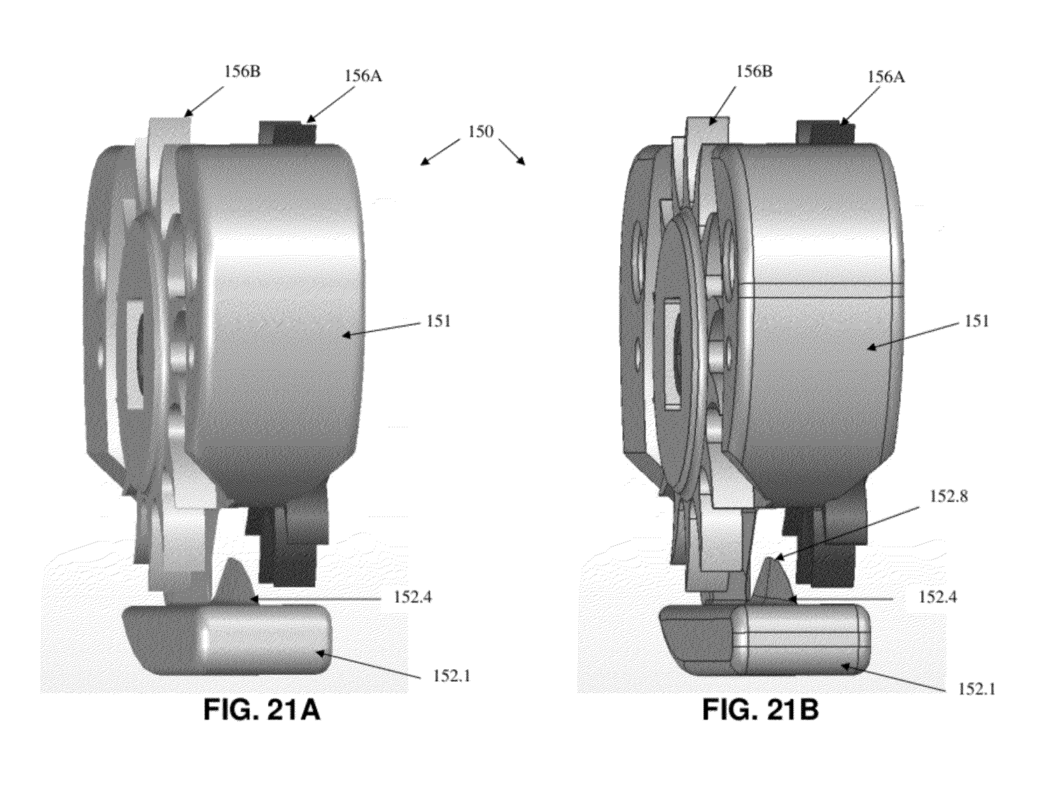

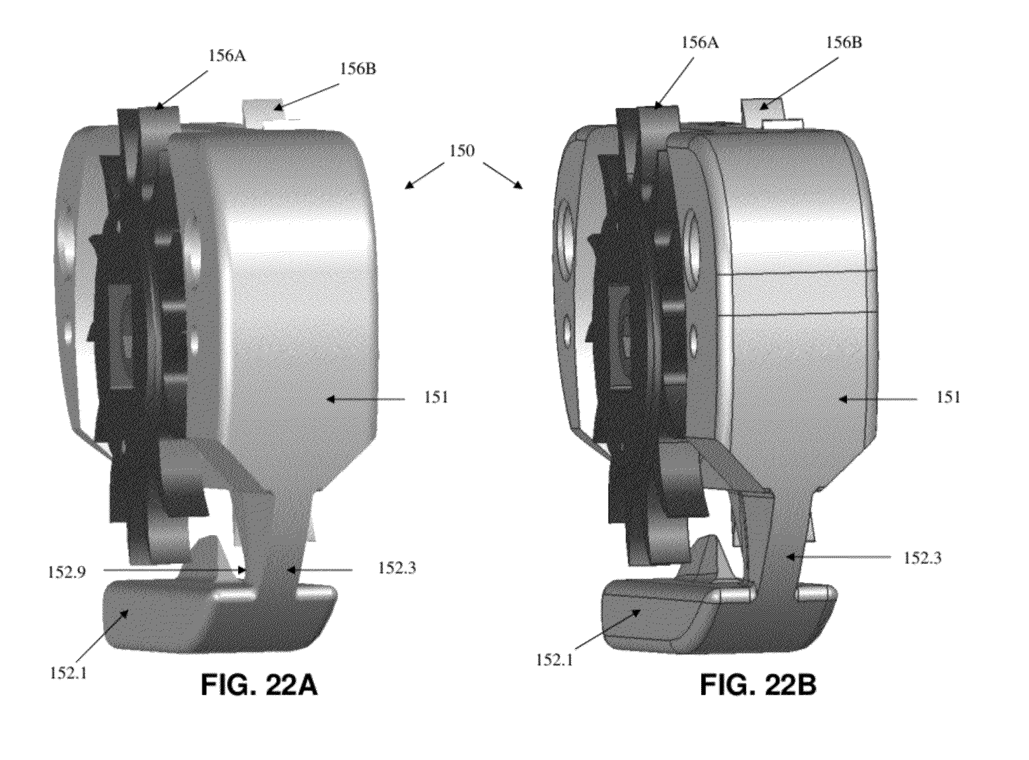

Referring to FIGS. 20, 21, and 22, cutting assembly 150 includes a supporting member 151 that is coupled to an apparatus such as apparatus 20. Support member 151 supports a pair of advancing wheels 156A and 156B, the teeth of which are on opposing sides of the support member 151. Front and rear housing covers 151.1 and 151.2, respectively, (not shown) shield the user from portions of the advancing wheels. A keel 152 extends downward from one side and toward the other side from support member 151. As best seen in FIGS. 21 and 22, a front splitting wedge 152.8 and a rear splitting wedge 152.9 extend from foot 152.1 of keel 152, and are generally placed between advancing wheels 156A and 156B.

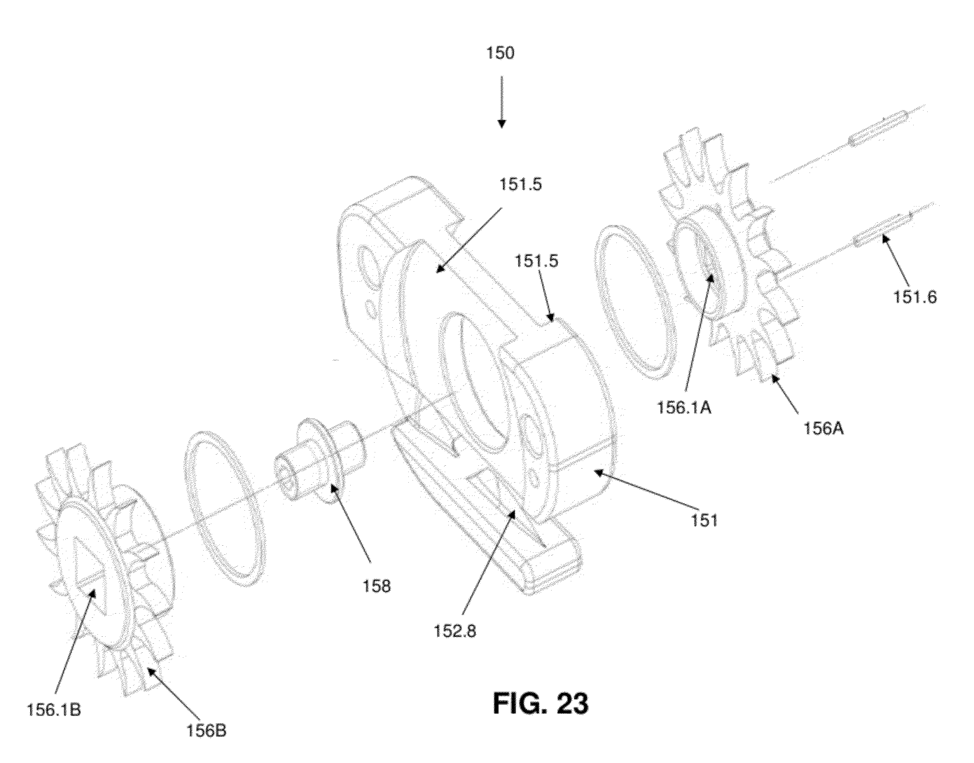

FIGS. 23, 24, and 25 depict the assembled elements that comprise a cutting assembly 150 according to one embodiment of the present invention. A central support member 151 supports a pair of advancing wheels 156A and 156B that are coupled together by a pair of pins 151.6. Each wheel includes a driven interface 156.1 that has a shape complementary with the shape of adaptor drive 46.3 of gear reduction assembly 40 (referring to FIG. 12). Referring to FIG. 25, this driven interface (shown in FIG. 20 as a substantially square interface) can drive either one of the advancing wheels. A hubscrew 158, which threadably couples to a threaded pocket of adaptor drive 46.3, can move toward either wheel along the drive axis, as permitted by the flange 158.1 located within a pocket 156.8 formed between wheels 156A and 156B.

As best seen in FIGS. 23 and 25, central support member 151 includes a pair of semi-circular pockets 151.5 scalloped onto either face. Each advancing wheel 156 is generally centered within a corresponding pocket 151.5. In some embodiments, central support member 151 does not include enclosures that extend around the advancing wheel, although the present invention does contemplate a multi-part support member 151 similar to housing 51 that at least partly encloses the advancing wheels.

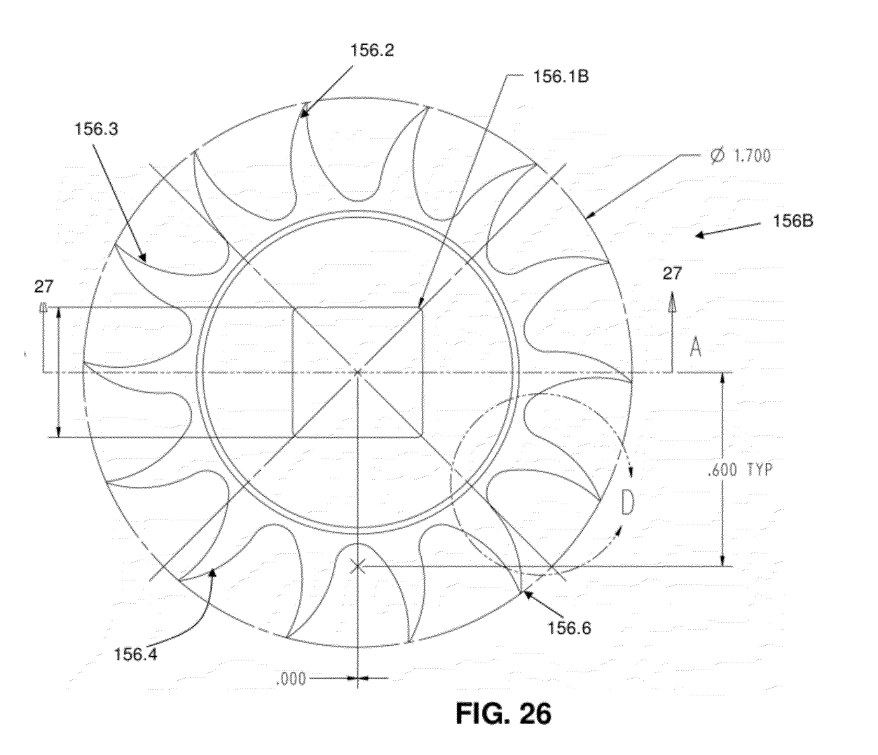

FIGS. 26, 27, and 28 show an advancing wheel 156b according to one embodiment of the present invention. In one embodiment, wheel 156a is identical to wheel 156b, although the present invention contemplates embodiments in which the wheels are different.

Wheel 156b includes a pattern of teeth 156.2 that extend in a repetitive pattern around the circumference of the wheel. Wheel 156b is adapted and configured to be rotated more than 360 degrees as the material is cut.

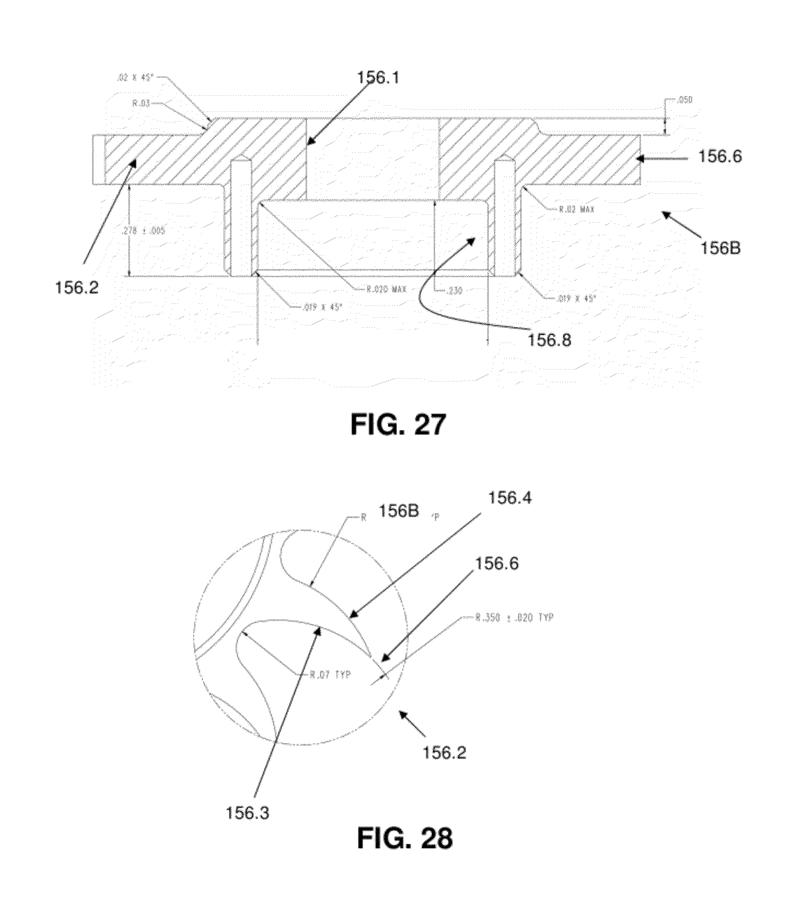

Referring to FIG. 28, the teeth of wheel 156b have a shape that is adapted and configured to have the tip 156.6 be the first point of contact with the cast material moving along path 150.1 as the wheel rotates. In one embodiment, the teeth comprise a convex side 156.4 and a concave side 156.3, the two sides meeting at apex 156.6. In another embodiment, the concave and convex sides are circular in shape and have the same radius. However, the present invention is not so limited and contemplates other shapes of teeth. As best seen in FIG. 27, each tooth of wheel 156B has a generally constant cross sectional shape, such that tip 156.6 engages with the material to be cut with line contact. However, the present invention is not so constrained, and further includes those embodiments in which the cross sectional shape of the tooth narrows, such that contact with the material is more concentrated with subsequently higher bearing stresses in the material when contacted with the tooth.

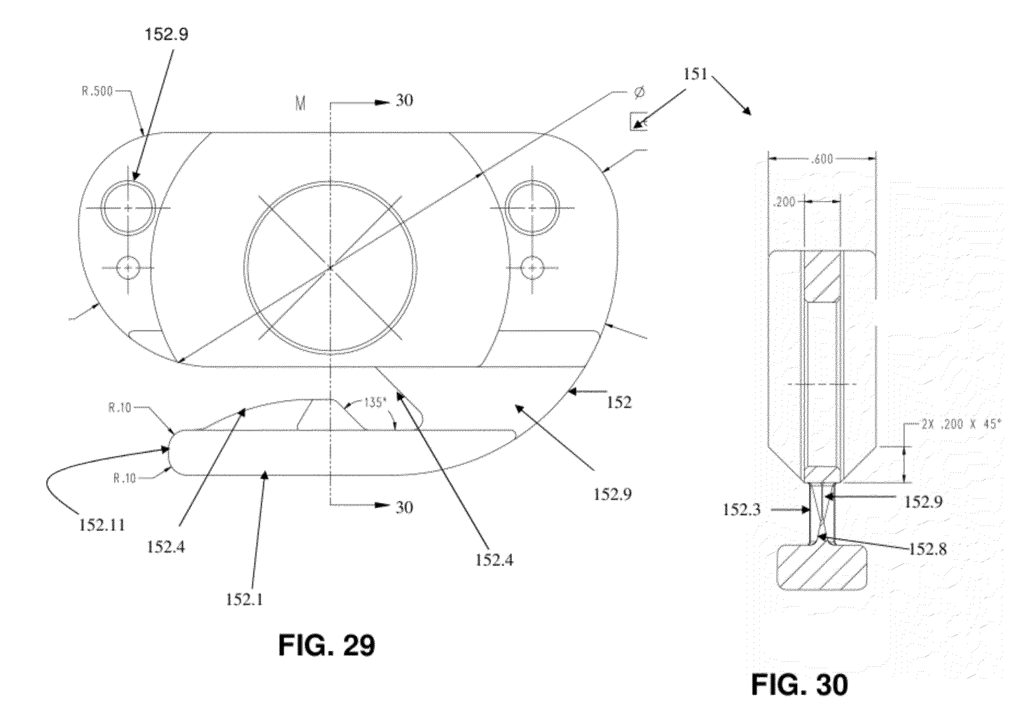

FIGS. 29 and 30 depict frontal and cross-sectional views, respectively, of a support member 151 according to one embodiment of the present invention. Support member 151 is coupled to the front of an apparatus such as apparatus 20 in a manner similar to that of cutting assembly 50.

Support member 151 includes a keel 152 that extends downward and from one side of support member 151. Keel 152 includes an elongated foot 152.1 supported in cantilever-fashion by arm 152.3. Keel 152 is elongated in a direction parallel with the movement of material. At the end of keel 152 opposite of arm 152.3 is a toe 152.11 that is adapted and configured to be located on the side of the material being cut opposite of advancing wheels 156. Extending upwardly from foot 152 is a front splitting wedge 152.8 which has a generally triangular or wedge-shaped cross-section (as best seen in FIG. 30). A sharp edge 152.4 extends along the top and front of wedge 152.8. As best seen in FIG. 29, the front edge of wedge 152.8 slopes upward from the top surface of foot 152.1 to a maximum height, and then falls back at a 135 degree angle to the top surface of foot 152.1. This sloping front edge and the lateral movement of the material being cut (from left to right in FIG. 29) permit sharp edge 152.4 of front wedge 152.8 to apply progressively increasing splitting pressure onto a surface of the material.

Support arm 152.3 further includes a rear wedging cross-section 152.9, and a sharp front edge 152.4. Material continuing to move under the action of wheel 156 is advanced over the sharp edge 152 of rear splitting wedge 152.9, such that the splitting action continues along the upper thickness of the material being cut.

Referring to FIGS. 25 and 20A, it can be seen that the material flow path 150.1 extends in a fashion similar to that of cutting assembly 50. The material to be cut or split is advanced from the right to left, as seen in FIG. 20A under the action of the teeth of the advancing wheels. As advancing wheels 156A and 156B rotate in a clockwise direction, the descending teeth contact and press into the material positioned along flow path 150.1. As the wheels continue to rotate, the teeth press progressively harder into the material, and further push and pull the material along the sharp edge 152.4 of front wedge 152.8. The sharp edge 152.4 splits the material, both by the piercing of the material with the sharp edge and the wedging action that occurs as the pierced material is pushed along the wedge shape. The material continues to move from right to left along flow path 150.5 under the action of the teeth of the advancing wheel, and subsequently encounters the sharp edge 152.4 of the rear wedge 152.9 of keel 152. Rear wedge 152.9 further includes a cross-sectional shape that increases similar in fashion to that of front wedge 152.8, to thereby continue the splitting and wedging-apart action on the material.

In one embodiment, wheels 156A and 156B have equal numbers of teeth, and further the teeth of each wheel are equally angularly spaced about the centerline of the wheel. In still further embodiments, the wheels are interlocked to each other such that the tooth pattern of one wheel is offset relative to the tooth pattern of the other wheel by one-half of the tooth spacing. This is best seen in FIGS. 20A and 20B. This interspaced interlocking of the wheels provides for more uniform movement of the material over the foot and onto the sharp edges.

In some embodiments of the present invention, the cutting assembly 150 includes a single wheel 156 that engages the material to be cut and moves it toward a sharp edge. However, yet other embodiments include a plurality of wheels, each of which has teeth in engagement with (or pressing against) the material to be cut. These wheels are spaced apart from one another so that their teeth engage different portions of the material being cut.

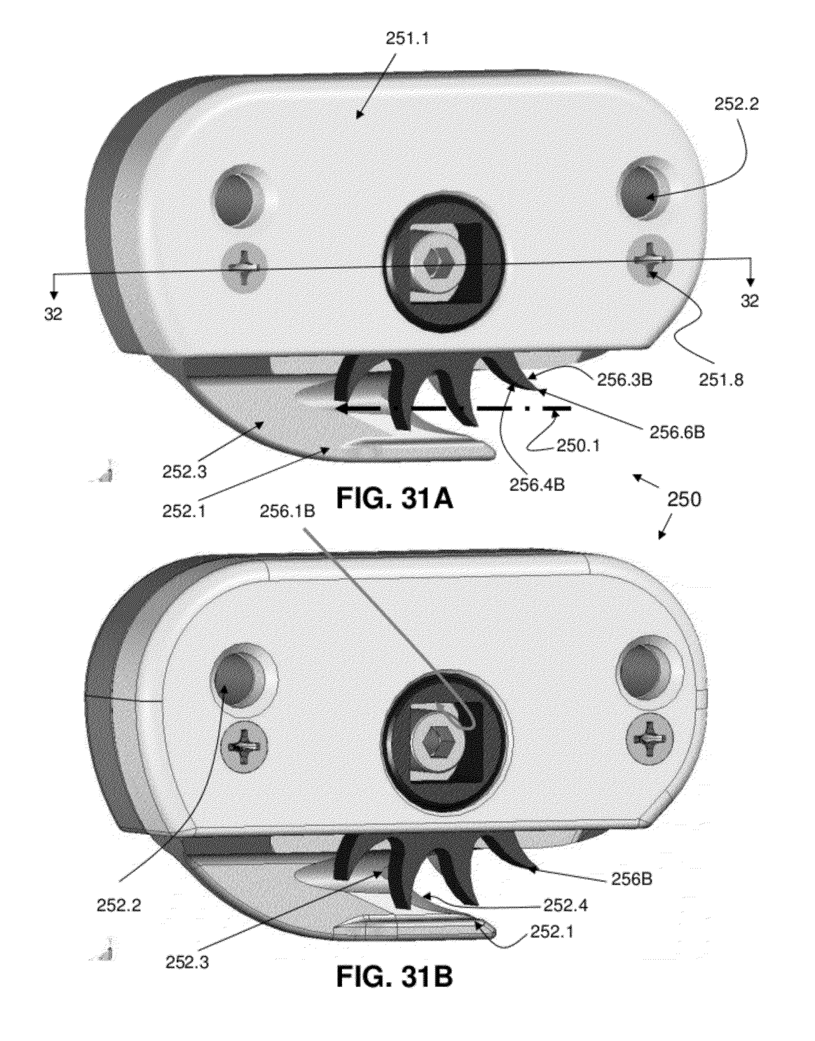

FIGS. 31, 32 and 33 depict different aspects of a cutting assembly 250 according to another embodiment of the present invention. Cutting assembly 250 operates in a manner similar to that of cutting assembly 150, except for differences that will be explained.

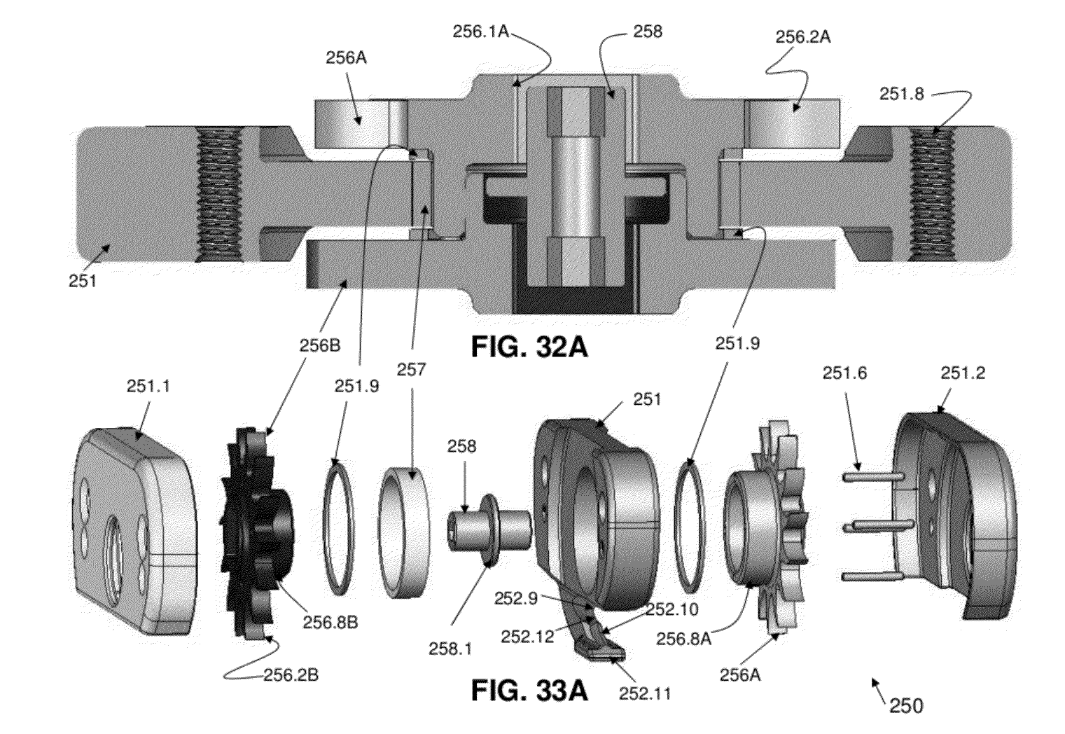

As best seen in FIGS. 31A and 32B, keel 252 extends downward from support 251 by an attachment arm 252.3. An elongated foot 252.1 extends laterally from arm 252.3 and ends in a toe 252.11 that is located generally under teeth 256.2.

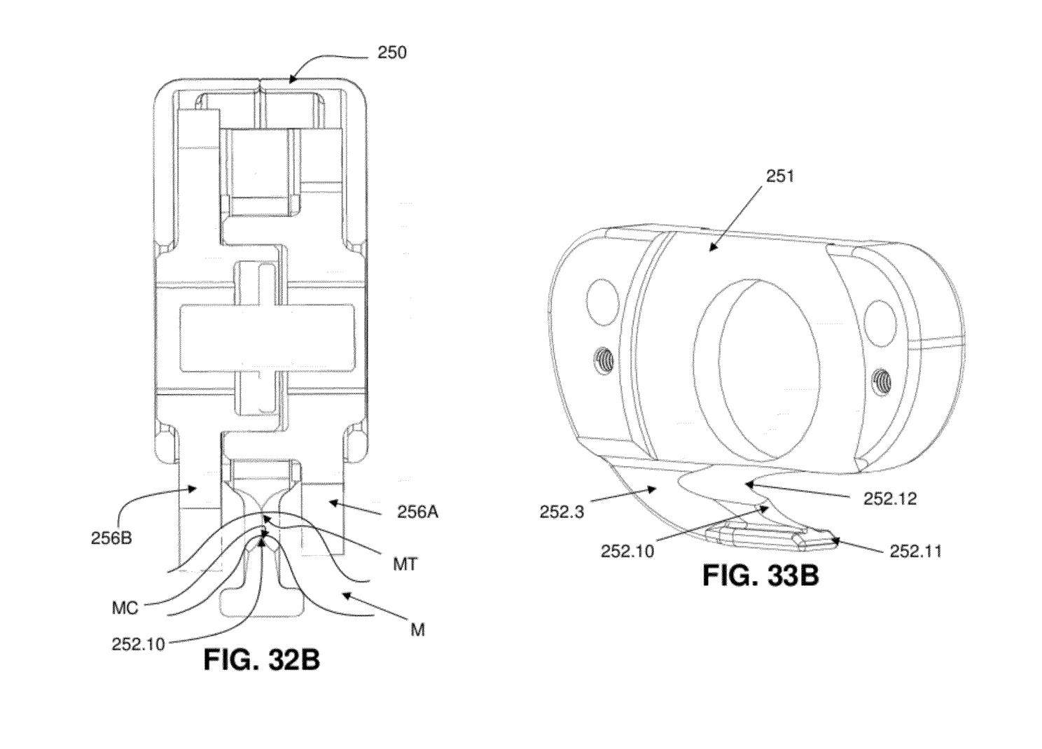

The rotational movement of teeth 256.2 moves the material along path 250.1 toward the sharp surface 252.4 of rear shearing wedge 252.9. In some embodiments, wedge 252.9 has a generally concave shape 252.12 that is open in the direction of accepting the material to be sheared and generally at the apex of the concavity (as best seen in FIG. 33B). Thus, wheels 256A and 256B press the material downward toward foot 252.1, bend the material over fin 252.10, and pull the material past the sharp edge 252.4 of rear cutting surface 252.9.

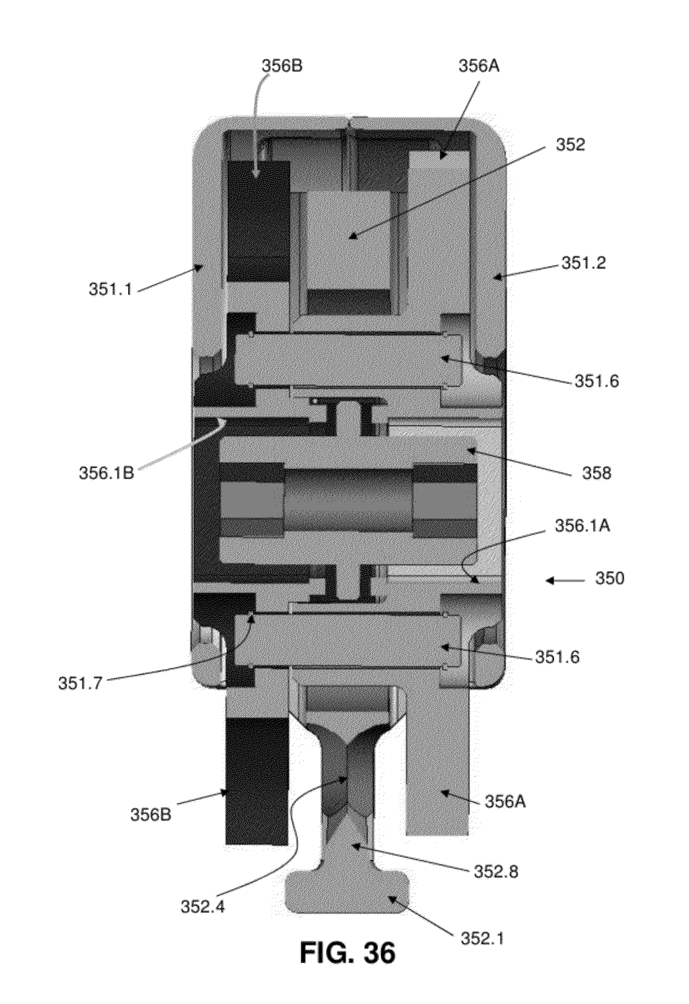

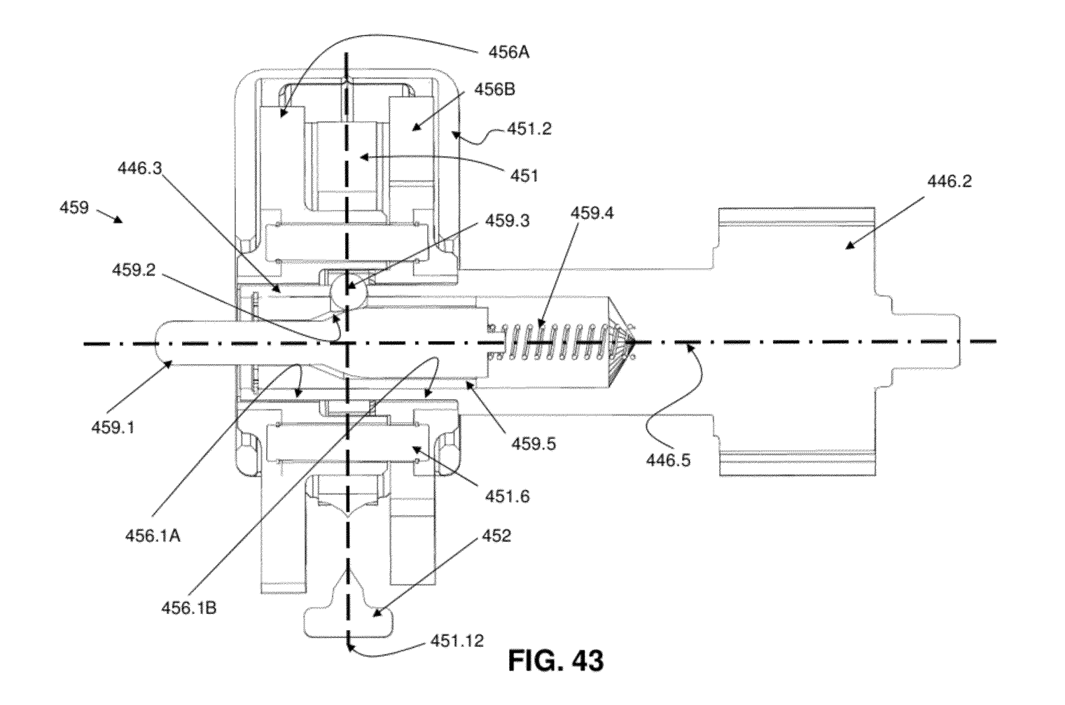

Foot 252.1 includes a fin 252.10 that rises upward from the top surface of the foot and extends toward the centerline of advancing wheels 256A and 256B. This fin 252.10 preferably comes to an apex (which can be seen in FIGS. 36 and 43 with regards to feet 352.1 and 452.1, respectively). Referring again to FIGS. 31B and 33, it can be seen that this apex of the fin slopes upward toward the rotational centerline. Therefore, as teeth 256.2A and 256.2B move the material in direction 250.1, the material to be cut is pressed downward by the teeth toward the top surface of foot 252.1, and the portion of material in between the teeth is pressed upward by the top surface (or apex) of fin 252.10. This motion places the material to be cut in bending, such that the topmost surface of the material tends to be in tension, and the bottommost surface (surface of the material in contact with the fin) is in a state of compression.

FIG. 32B is a cross sectional view of a cutting assembly 250 that is cutting a portion of a material M. The material M is being moved toward the shearing surfaces of foot 252.1 in direction 250.1 by the action of the teeth of advancing wheels 256A and 256B. The apex of the teeth are generally in pressing contact with the material M, and in some embodiments can puncture the material. The material has been drawn by the teeth over the toe 252.11 (not shown in FIG. 32B), and a portion of material is on top of raised fin 252.10 of foot 252.1. Fin 252.1 presses against the underside of material M. On either side of the fin the teeth of the advancing wheels push down on the material M, such that the material is bent in a downward U-shape. This bending places the top surface MT in tension, and the bottom surface MC in compression. This increased state of stress within the material reduces the amount of shearing force that needs to be applied by the sharp edge 252.4 within the concave cutting surface 252.12.

Cutting assembly 250 further includes a plain bearing 257 located between a cylindrical alignment pilot 256.8A and an inner diameter of support structure 251. A second cylindrical alignment pilot 256.8B is received within first pilot 256.8A, as best seen in FIG. 32. Bearing 257 is preferably fabricated from a lubricious material (or has a lubricious coating on the material), and in one embodiment is fabricated from Polylube® MRP material provided by Polygon Company. Bearing 257 prevents galling between the outer diameter of pilot 256.8A and the inner diameter of support structure 251. FIG. 33 further shows a pair of washers 251.9 located on either side of support 251, and providing spacing and bearing functions pertaining to translational movement of wheels 256A and 256B along their rotational axis. Four alignment pins 251.6 limit movement of wheel 256A relative to wheel 256B. Further, these pins 251.6 impart driving torque from the driven wheel (i.e., wheel 256A in FIGS. 31A, 31B, and 32) to the other (or front) wheel 256B. As stated previously for cutting assembly 50, this driving torque is imparted to wheel 256A by the driving surface 256.1A from a driving member such as driving member 46.3 (as seen in FIG. 12).

Wheels 256A and 256B include teeth that are oriented for advancing the material to be cut in a manner different than wheels 56 and 156. Referring first to FIGS. 20A and 20B, it can be seen that wheels 156 include teeth that have a concave surface on one side, and a convex surface on the other side. As the wheels 156 turn in their normal manner, the concave side leads the convex side in advancement along direction 150.1. Thus, as teeth 156 rotate clockwise from a position closest to foot 152.1 (as viewed in FIG. 20A), the concave side of a tooth 156.2 grabs and “scoops up” the material being cut, and in some embodiments can act to lift the material as it is being sheared in rear cutting surface 152.9.

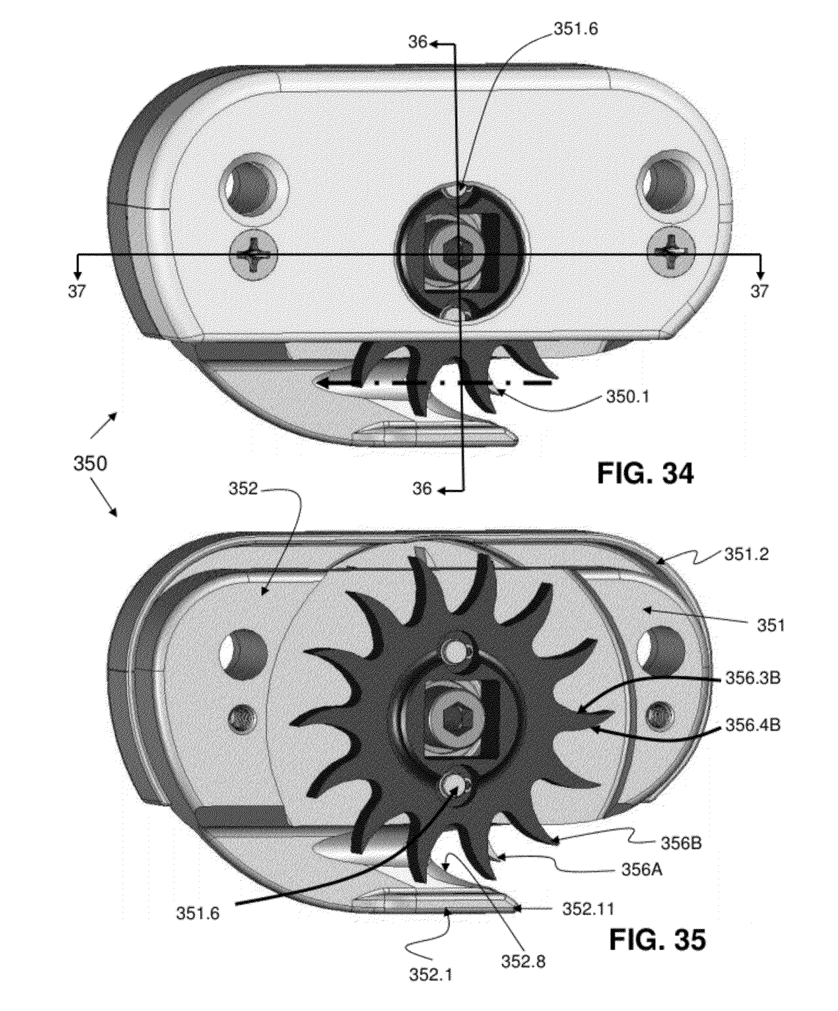

In contrast, the orientation of the teeth 256.2 of cutting assembly 250 are oriented in an opposite direction. As wheel 256 rotates in its normal manner, the leading edge of a tooth is the convex side 256.4, and the trailing side is the concave side 256.3. Referring to FIG. 31A, as wheel 256B rotates to move material in direction 250.1, there is less tendency to lift up the material, since the leading of the tooth is a convex surface. Therefore, as wheel 256 rotates, the convex side of the tooth nearest the apex first contacts the material, followed by the concave side which trails the apex. As the tooth continues to rotate and move upward and away from foot 252.1, the convex leading edge of the tooth facilitates the sliding off of the punctured material from the tooth. FIGS. 34-40 depict various views of a cutting assembly 350 according to another embodiment of the present invention. As will be appreciated by one of ordinary skill in the art, cutting assembly 350 is similar to cutting assemblies 50, 150, and 250, except for the differences that will be shown and described.

Cutting assembly 350 includes a pair of advancing wheels 356A and 356B that include a modified means of providing torque from the wheel driven by the gear train to the other wheel. Referring to FIG. 35, driving torque from the gear train is provided to wheel 356A. Wheel 356A subsequently drives outermost wheel 356B.

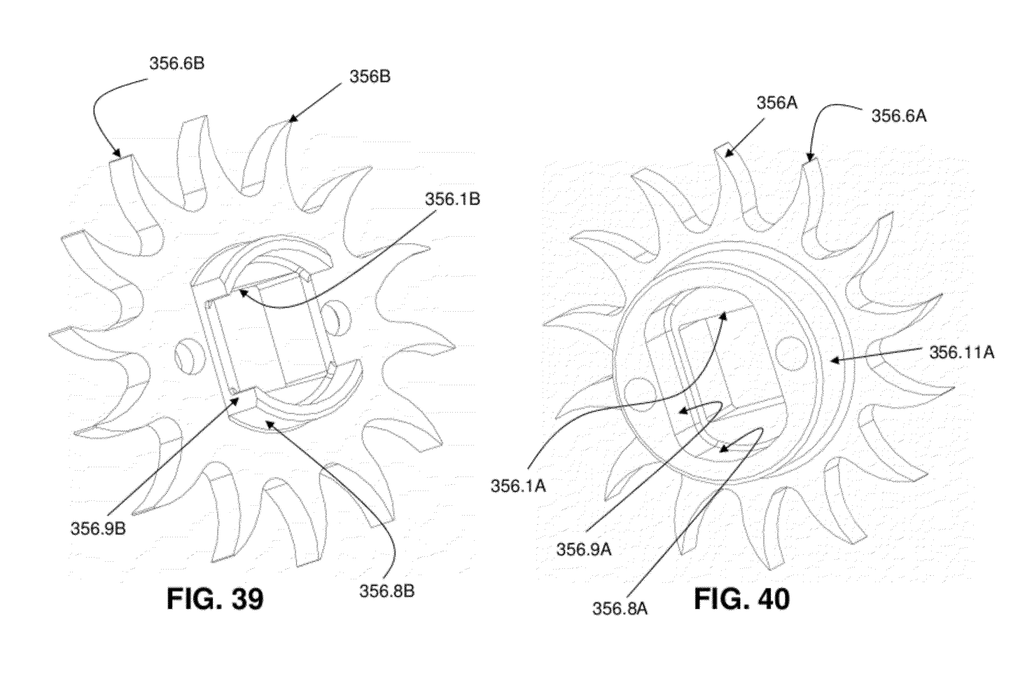

Referring to FIGS. 39 and 40, the hub of wheel 356A includes an outermost diameter 356.11 that is received generally within the inner diameter 351.10 of support 351 (as best seen in FIG. 38). Referring again to FIG. 40, wheel 356A further includes an inner diameter 356.8A that receives within it a piloting outer diameter 356.8B of the hub of wheel 356B (as best seen in FIG. 39). However, the pilot of wheel 356B is squared off on two edges so as to form a pair of partly cylindrical ears 356.9B. The squared off surfaces of these ears are received within the mating flat surfaces 356.9A of wheel 356A. By the placement of these ears within a complementary-shaped pocket, a driving torque applied to driving surface 356.1A is transmitted by surfaces 356.9A to the ears 356.9B of wheel 356B. Likewise, if cutting assembly 350 is placed on apparatus 20 in the opposite direction (so as to cut material in a direction opposite that as indicated by arrow 350.1), then the driving torque imparted by the gear train to driven interface 356.1B will be transmitted by ears 356.9B to the walls of pocket 356.9A of wheel 356A.

Therefore, it can be seen that cutting assembly 350 includes a pair of advancing wheels that are keyed or interlocked to each other so as to impart torque directly from the structural web of one wheel to the structural web of the adjacent wheel. It is not necessary to transmit torque by pins 351.6. Instead, these pins can maintain the location of one wheel relative to the other and prevent sliding apart along the axis of rotation. As seen best in FIGS. 35 and 38, each pin 351.6 is locked in place by a pair of spring clips 35.7 that fit within grooves on either end of the pin.

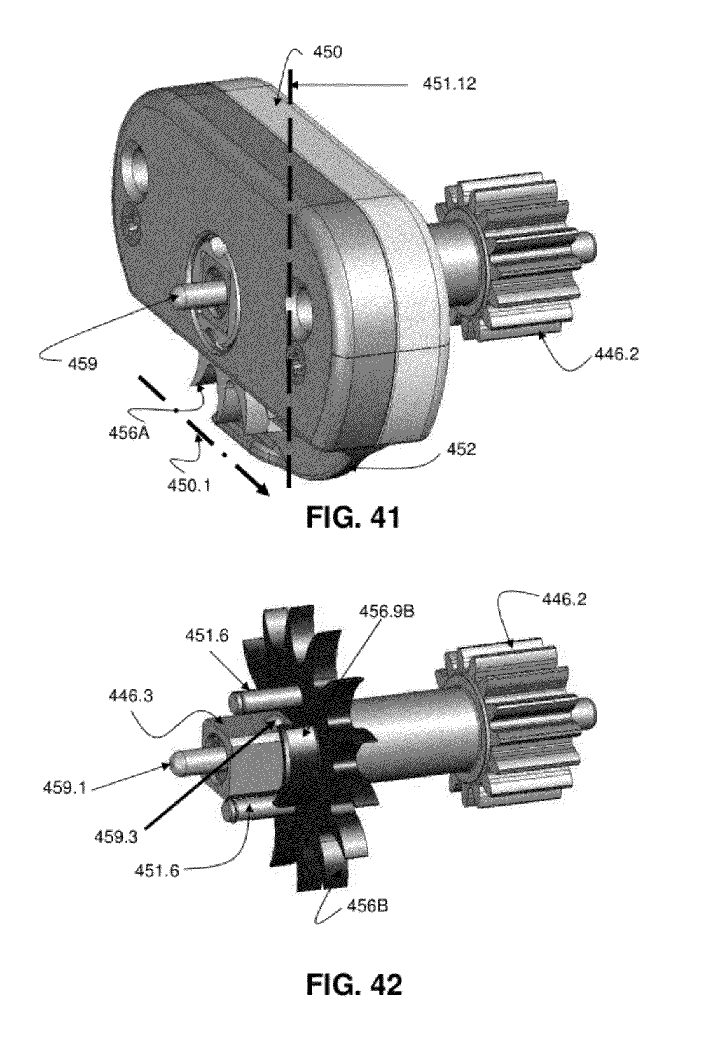

FIGS. 41-44 depict various views of a portion of an apparatus according to another embodiment of the present invention. FIG. 41 shows a cutting assembly 450 placed in driving engagement with a drive adapter 446.3 extending on one end of the shaft that also includes driven gear 446.2 of a pair of pinion gears (similar to the pinion gears 46.2 and 46.1 shown in FIGS. 12-15). Cutting assembly 450 is similar to cutting assemblies 50, 150, 250, and 350, except as will be shown and described.

FIG. 42 shows a portion of the apparatus of FIG. 41, except with housing halves 451.2 and 451.1 removed, and also with support structure 451 (including the keel and the foot) removed. It can be seen that pinion gear 446.2 is located on one end of a shaft, with the other end of the shaft including a squared-off driving section 446.3. Driving member 446.3 has a length that is sufficient for it to extend completely through the hubs of the advancing wheels 456A and 456B. As best seen in FIG. 43, and as can be appreciated from FIG. 42, the driving portion 446.3 extends within advancing wheel 456A. Therefore, drive adapter 446.3 can directly drive both wheels 456B and 456A.

Referring to FIGS. 42 and 43, it can be seen that these figures show cutting assembly 450 coupled to the drive adapter 446.3 in a second position that is different than the position of the cutting head 350. FIG. 42 shows that the wheel closest to pinion gear 446.2 is wheel 456B, which includes a pair of driving ears 456.9B. In a manner similar to that discussed with regards to cutting assembly 350, these drive ears are received within a complementary-shaped driving pocket of wheel 456A. Thus, in cutting assembly 450 the wheels 456 have features that interlock them so that they rotate in unison, and further these features (such as the ears) permit the transmission of torque. In addition, torque is transmitted directly to each wheel by the driving element 446.3 from pinion gear 446.2

In comparing FIGS. 34 and 41, it is evident that cutting assemblies according to various embodiments of the present invention can be located on a driving shaft in either of two positions. FIG. 34 shows a first position in which the flow of material 350.1 is from right to left, and in which advancing wheel 356B is the front-most advancing wheel and wheel 356A is closest to the pinion gear.

FIG. 41 shows a cutting assembly 450 in which the assembly is oriented in a second position. This position is revolved about axis 451.12 by one-half turn. Referring to FIG. 43, it is seen that interchangeability axis 451.12 is generally perpendicular to rotational axis 446.5 of the advancing wheels.

Returning to FIG. 41, it is seen that after this change in position, the flow of material 450.1 is generally now from left to right, and further that the front-most advancing wheel is wheel 456A, and wheel 456B is comparatively closer to pinion 446.2. Thus, the mounting and drive features of the cutting assemblies 50, 150, 250, 350, and 450, are adapted and configured to provide two different cutting directions based on placement of the cutting head in either of the two different positions.

Cutting assembly 450 and driving member 446.3 are further adapted and configured to provide easy releasability of cutting assembly 450 from drive adapter 446.3, and subsequently easy reattachment in the same position (such as replacing a worn cutting edge) or in the other position (such as for cutting in the other direction). As best seen in FIGS. 42, 43, and 44, driving member 446.3 and cutting assembly 450 are adapted and configured to accommodate means 459 for readily disengaging the cutting assembly from the driving member. In one embodiment, means 456 includes a push button 459.1 located within a collar 459.5, both of which are in a bore of driving member 446.3. A spring 459.4 biases push button 459.1 outward from the front face of cutting assembly 450.

Referring to FIG. 43, collar 459.5 and driving member 446.3 are adapted and configured to receive a ball 459.3. Ball 459.3 is pushed outwardly by a sloping, transitional detent 459.2 of button 459.1. Therefore, button 459.1 is spring loaded to push ball 459.3 outward past the outermost surface of driving member 446.3 (as best seen in FIGS. 42 and 43). This ball extends between the hubs of wheels 456A and 456B. Any attempt to slide the wheels off of driving member 446.3 will be obstructed by interference between the ball 459.3 and the innermost hub (the hub of wheel 456A in FIG. 43). Therefore, the location of cutting assembly 450 along rotational axis 446.5 is established by interference of the ball and the wheel hub, and further by the other end of the hub pressing against a shoulder of driving element 446.3.

Removal of cutting assembly 450 is easily accomplished by pushing button 459.1 inward, such that ball 459.3 drops to a lower position and no longer interferes with the hub of the innermost advancing wheel. When ball 459.3 is in the lower position, cutting assembly 450 can be removed by translating it forward in a direction parallel to the rotational axis. Likewise, to engage cutting assembly 450 onto driving member 446.3, the pushbutton is again pushed inward to lower the position of the ball, and the cutting head can be reinserted over the driving member and the dowels 34.2 (as seen in FIG. 6).

Other embodiments of the present invention contemplate other means for readily engaging and disengaging the cutting assembly from the driving member. As seen with regards to other cutting heads described herein, another means for readily engaging and disengaging the cutting assembly is a central socket screw 58 or 158 that threadably engages a set of interior threads within driving member 46.3. Further, yet other embodiments of the present invention contemplate a spring loaded, bayonet-type of engagement mechanism such as those used in some electrical connectors. Preferably, the engaging and disengaging means does not require a separate tool, and further in some embodiments can be accomplished with a single hand of the user.

While the inventions have been illustrated and described in detail in the drawings and foregoing description, the same is to be considered as illustrative and not restrictive in character, it being understood that only the preferred embodiment has been shown and described and that all changes and modifications that come within the spirit of the invention are desired to be protected.

1. An apparatus for cutting a layer of material, comprising:
an electric motor having an output speed and an output torque;
a gear train receiving the output speed and output torque of said motor, said gear train including a driving member operating at a driving speed lower than the output speed and a driving torque higher than the output torque;
a first wheel including a first plurality of teeth arranged about a rotational axis, said first wheel being rotationally driven by said driving member;
a second wheel including a second plurality of teeth arranged about a rotational axis;
a foot having a top surface and a sharp edge, the sharp edge being located between said first wheel and said second wheel; and
wherein rotation of said first wheel and said second wheel more than 360 degrees about the rotational axis press the material against the top surface and move the material toward the sharp edge.

2. The apparatus of claim 1 wherein said second wheel is driven by said first wheel.

3. The apparatus of claim 1 wherein said second wheel is driven by said driving member.

4. (canceled)

5. The apparatus of claim 4 wherein the first plurality and the second plurality are each equally spaced apart circumferentially about the axis.

6. The apparatus of claim 4 wherein said first wheel and said second wheel rotate in unison with one another.

7. The apparatus of claim 4 wherein the angular positions of each of the first plurality of teeth are in-between the angular positions of each of the second plurality of teeth.

8. The apparatus of claim 1 wherein the sharp edge has a concave shape open for receiving the moving material.

9. The apparatus of claim 1 the top surface includes a raised central fin aligned with the sharp edge.

10. A method for cutting a layer of material, comprising:
providing a first plurality of teeth arranged about an axis, a second plurality of teeth arranged about the axis, and a foot having a shearing surface and a top surface, the shearing surface being between the first plurality and the second plurality;
engaging the material with at least one tooth from the first plurality and at least one tooth from the second plurality;
continuously rotating the first plurality of teeth and the second plurality of teeth about the axis;
moving a portion of the engaged material by said rotating toward the shearing surface and above the top surface; and
cutting the material with the shearing surface by said rotating.

11. The method of claim 10 wherein said engaging is by puncturing the surface of the material with the teeth.

12. The method of claim 10 wherein the foot is elongated in a direction substantially perpendicular to the axis.

13. The method of claim 10 wherein the material is a orthopedic cast on a limb of an animal, the foot has a bottom surface, and which further comprises placing the foot between the limb and the cast before said engaging.

14. The method of claim 10 wherein the top surface includes a raised central fin and which further comprises bending the material on the fin by said moving and before said cutting.

15. The method of claim 10 which further comprises tensioning the surface of the material closest to the axis before said cutting.

16. An apparatus for cutting a layer of material, comprising:
an electric motor;
a wheel including a plurality of teeth equally spaced about a rotational axis, each said tooth having a concave side and a convex side, said wheel being rotationally driven in a direction by said electric motor, the convex side of each said tooth leading the concave side of the same said tooth during rotation in the direction; and
an elongated foot having a top surface and a sharp edge, said foot being elongated in a direction generally perpendicular to the axis, one end of said foot including a toe adapted and configured for being placed on a side of the material opposite of said wheel, the other end of said foot including the sharp edge;
wherein during rotation the teeth press the material against the top surface and move the material from the toe toward the sharp edge.

17. The apparatus of claim 16 wherein said wheel is a first wheel having a first plurality of teeth and which further comprises a second wheel including a second plurality of teeth equally spaced about the rotational axis, each said tooth of the second plurality having a concave side and a convex side, said second wheel being rotationally driven in the same direction, the convex side of each said tooth leading the concave side of the same said tooth during rotation in the direction.

18.-32. (canceled)

33. The apparatus of claim 17 wherein the number of first teeth is equal to the number of second teeth, and said first wheel and said second wheel are interlocked to each other such that each of said first teeth is aligned inbetween a pair of said second teeth.

34. The apparatus of claim 16 which further comprises a plurality of wheels each including a plurality of teeth equally spaced about a rotational axis, each said tooth having a concave side and a convex side, each said wheel being rotationally driven in a direction by said electric motor, the convex side of each said tooth leading the concave side of the same said tooth during rotation in the direction.

35. (canceled)

36. A method for cutting a layer of material, comprising:
providing a first plurality of teeth arranged about an axis, a second plurality of teeth arranged about the axis, and a foot having a cutting surface and a top surface, the cutting surface being between the first plurality and the second plurality;
engaging the material with at least one tooth from the first plurality and at least one tooth from the second plurality;
continuously rotating the first plurality of teeth and the second plurality of teeth about the axis;
moving a portion of the engaged material by said rotating toward the cutting surface and above the top surface; and
cutting the material with the cutting surface by said rotating.

37. The method of claim 36 wherein the material is a orthopedic cast on a limb of an animal, the foot has a bottom surface, and which further comprises placing the foot between the limb and the cast before said engaging.
